(19) United States
(12) Patent Application Publication
(10) Pub. No.: US 2024/0299379 A1
(43) Pub. Date: Sep. 12, 2024

(54) COMPOSITIONS OF CLOFAZIMINE AND AMIKACIN FOR PULMONARY ADMINISTRATION IN THE TREATMENT OF RESPIRATORY DISEASES

(71) Applicant: BOARD OF REGENTS, THE UNIVERSITY OF TEXAS SYSTEM, Austin, TX (US)

(72) Inventors: Hugh D.C. SMYTH, Austin, TX (US); Ashlee D. BRUNAUGH, Austin, TX (US)

(21) Appl. No.: 18/566,008

(22) PCT Filed: Jun. 1, 2022
(86) PCT No.: PCT/US2022/031852

§ 371 Date: Nov. 30, 2023

Related U.S. Application Data
(60) Provisional application No. 63/195,388, filed on Jun. 1, 2021.

(51) Int. Cl. A61K 31/498; A61K 9/00; A61K 31/7036

(52) U.S. Cl. CPC A61K 31/498; A61K 9/0075; A61K 31/7036

(57) ABSTRACT

The present disclosure provides pharmaceutical compositions of clofazimine and amikacin that may be administered via inhalation. These compositions may be used to treat one or more diseases or disorders such as a bacterial infection or other lung diseases or disorders. These compositions may be made using a spray drying method that results in deposition of the clofazimine onto the amikacin.

This application claims the benefit of priority to U.S. Provisional Application No. 63/195,388, filed on Jun. 1, 2021, the entire contents of which are hereby incorporated by reference.

This invention was made with government support under Grant no. F31 HL146178 awarded by the National Institutes of Health. The government has certain rights in the invention

### BACKGROUND

### 1. Field

The present disclosure relates generally to the field of pharmaceuticals and pharmaceutical manufacture. More particularly, it concerns compositions and methods of preparing a pharmaceutical composition containing clofazimine and amikacin.

### 2. Description of Related Art

Currently, the treatment options for bacterial infections such as infections of mycobacteria such as Mycobacterium abscessus (Mab), Mycobacterium avium, and Mycobacterium tuberculosis lung infections, as the emergence of drug resistance bacterial populations has become a global healthcare concern. For example, pulmonary Mab infections treatment guidelines recommend a two-year regimen of a combination of four antibiotics that are associated with numerous systemic toxicities that require regular monitoring. Despite this intensive therapeutic regimen, treatment is Mab is successfully in only 30% of cases (van Dorn, 2017), and chronic Mab infection is associated with progressive decrease in lung function in CF patients (Esther et al., 2010). More worrisome still, is that unlike other non-tuberculous mycobacterium (NTM), the majority of highly drug resistant Mab infections appear to derive from human-to-human transmission (Bryant et al., 2016). Mab exhibits several innate and acquired drug resistance mechanisms. Poor penetration of antibiotics is noted due to the lipid rich content of the cell wall (Flores, 2005), while the heterogeneity in cell division timing and morphology of daughter cells results in populations that exhibit mixed susceptibility to antibiotics. (Aldridge et al., 2012; Connolly et al., 2007) The alveolar macrophage is ground zero for the initiation and propagation of pulmonary mycobacterial infections, and mycobacteria have at their disposal a number of mechanisms to enable survival within the harsh intracellular environment, including inhibition of phagosome-lysosome fusion (Russell et al., 2009; Pieters, 2008; Frehel et al., 1986), inactivation of peroxidases (Pieters, 2008) and inhibition of phagosome acidification (Hmama et al., 2015; Crowle et al., 1991; Sturgill-Koszycki et al., 1994). Both smooth (S)- and rough (R)-morphologies of Mab have demonstrated the ability to establish intracellular infections, with the R-type exhibiting increased number of bacilli per phagosome which may be correlated to the more aggressive and virulent nature of this sub-type. (Roux et al., 2016) Like other mycobacterial lung diseases, Mab infection results in the formation of necrotizing granulomas (Tomashefski et al., 1996) which may harbor persistent populations of extracellular mycobacteria due to reduced vascularization and poor drug penetration of these sites (Dartois, 2014). Mab also exhibits a propensity for cording behavior. In zebrafish embryos, Mab R-type variants released from macrophages after apoptosis exhibited increased cording. (Bernut et al., 2014) The large size of the resulting extracellular aggregates prevented macrophage phagocytosis and thereby increased extracellular bacteria replication and abscess formation. These data indicate that Mab infections likely include populations of both extracellular and intracellular bacilli, both of which must be addressed for successful infection resolution.

Treatment of Mab lung infections with inhaled antibiotics may be a viable alternative to systemic drug therapy, as it would enable higher drug levels at the primary site of infection and reduce systemic drug exposure and therefore side effects. Nebulization of intravenous amikacin (AMK) formulations have been utilized as an off-label treatment for NTM lung infections, including Mab (Floto et al., 2016). More recently, the FDA approved a nebulized liposomal formulation of AMK for the treatment of refractory M. avium complex (MAC) lung disease. (Food and Drug Administration, 2018) This formulation is designed to increase macrophage uptake and biofilm penetration of AMK. (Zhang et al., 2018) However, the clinical benefits of liposomal AMK appear to be limited to the treatment of MAC lung disease. (Olivier et al., 2017) Additionally, pan-aminoglycoside resistant strains of Mab have emerged (Luthra et al., 2018), which is likely to reduce the utility of inhaled liposomal amikacin. Recently, promising in vitro synergistic activity against Mab was observed when AMK was used in combination with clofazimine (CFZ). (van Ingen, et al., 2012; Shen et al., 2010) This synergistic activity may serve as a mechanism to overcome highly drug resistant strains of Mab. CFZ is an existing, off-patent antibiotic that was originally developed for the treatment of tuberculosis but is now used for leprosy. The oral formulation of CFZ (available through compassionate-use regulatory pathways in the US) is recommended in Mab guidelines based upon its in vitro activity against Mab. (Floto et al., 2016, Daley et al., 2020) However, the wide-spread utilization of CFZ has been severely limited by the toxicities associated with the oral form of the drug. In a clinical trial examining the use of oral CFZ for M. tuberculosis (Mtb) (van Deun et al., 2010), adverse drug reactions were frequent, with 47.5% of the patient population reporting at least one adverse drug effect, and vomiting being the most frequent. Reddish-orange discoloration of the skin and ichthyosis is common, which can persist long after treatment discontinuation, and is distressing to patients. Most significantly, prolonged therapy with oral clofazimine can result in severe or fatal intestinal enteropathy and bleeding because of crystallization of the drug in the small bowel mucosa (Szeto et al., 2016; Arbiser et al., 19f95). Oral administration is also limited by the slow onset of action. At least 30 days of administration is necessary to reach steady-state concentrations, necessitating the use of large loading doses (Holdiness et al., 1989), and a delay in bactericidal activity occurs for up to two weeks after oral dosing, regardless of the dose administered (Swanson et al., 2015). Recent efforts have been made to develop an inhaled formulation of CFZ to avoid the issues associated with the oral form of the drug. Banaschewski et al. (2019) published results for an in vivo evaluation of a nebulized CFZ suspension in acute and chronic models of MAC and Mab lung infection. Despite promising results in the reduction of bacterial loads in chronic MAC infection, extremely limited reductions were noted in the treatment of a chronic Mab infection versus untreated controls.

Both the clinical results for inhaled liposomal AMK and the in vivo results for inhaled CFZ indicate that inhalation of a single agent may be insufficient to overcome highly drug resistant Mab infections. In contrast, simultaneous delivery and co-deposition of CFZ and AMK in the lung may enable synergistic antibacterial activity similar to what is noted in vitro and may reduce the drug payload required for each compound. Inhaled fixed-dose combinations (I-FDCs) have been used to good effect in the treatment of asthma and COPD, and it's likely that these benefits could be translated to the treatment of pulmonary mycobacterial infections.

### SUMMARY

In some aspects, the present disclosure provides compositions of clofazimine and amikacin. These compositions may be formulated to allow the administration of the two drugs together as a single dose. In some aspects, the present disclosure provides pharmaceutical compositions comprising a combined dose of:
(A) clofazimine or a pharmaceutically acceptable salt thereof; and
(B) amikacin or a pharmaceutically acceptable salt thereof.

In other aspects, the present disclosure also provides compounds that comprise a single agent that may be formulated into a device that allows the administration of both agents either sequentially or simultaneously.

In some embodiments, the clofazimine and the amikacin are prepared by physical mixture. In some embodiments, the clofazimine has been processed before mixing with amikacin by milling. In some embodiments, the amikacin has been processed before mixing with clofazimine by milling. In other embodiments, the amikacin or the clofazimine has been prepared by air jet milling. In other embodiments, the amikacin and the clofazimine have been processed in a single mixture by spray drying. In some embodiments, the pharmaceutical composition results in a drug particle comprising both amikacin and clofazimine.

In some embodiments, the pharmaceutical compositions are formulated for administration via inhalation. In some embodiments, the clofazimine is deposited on the surface of the amikacin. In some embodiments, the less than 25% of the clofazimine is present as a crystal with an aspect ratio of not less than 0.75. In some embodiments, less than 10% of the clofazimine is present as elongated needle-crystals. In some embodiments, less than 5% of 20 the clofazimine is present as elongated needle-crystals. In some embodiments, less than 2% of the clofazimine is present as elongated needle-crystals. In some embodiments, less than 1% of the clofazimine is present as elongated needle-crystals. In some embodiments, the amikacin is in the amorphous form. In some embodiments, the amikacin is in the amorphous form and the clofazimine is in the crystalline form.

In some embodiments, the pharmaceutical composition comprises from about 1% w/w of the clofazimine to about 75% w/w of clofazimine. In some embodiments, the pharmaceutical composition comprises from about 2.5% w/w to about 65% w/w of clofazimine. In some embodiments, the pharmaceutical composition comprises from about 5% w/w to about 60% w/w of clofazimine.

In some embodiments, the pharmaceutical composition is formulated without any excipients. In other embodiments, the pharmaceutical composition is formulated with one or more excipients. In some embodiments, the pharmaceutical composition comprises a first excipient. In some embodiments, the first excipient is a surfactant. In some embodiments, the surfactant is a non-ionic surfactant. In some embodiments, the surfactant comprises a polymer conjugated sorbitan. In some embodiments, the surfactant comprises a fatty acid group. In some embodiments, the surfactant is a polysorbate. In some embodiments, the surfactant is polysorbate 80. In some embodiments, the pharmaceutical composition comprises an amount of the first excipient from about 0.1% w/w to about 10% w/w of the first excipient. In some embodiments, the amount is from about 0.5% w/w to about 5% w/w of the first excipient. In some embodiments, the amount is from about 1% w/w to about 2.5% w/w of the first excipient.

In some embodiments, the pharmaceutical composition further comprises a second excipient. In some embodiments, the second excipient is a lipid. In some embodiments, the second excipient is a phospholipid. In some embodiments, the second excipient comprises one or more fatty acid groups. In some embodiments, the second excipient comprises two or more fatty acid groups. In some embodiments, the second excipient comprises a phosphate group. In some embodiments, the second excipient comprises a negatively charged phosphate group. In some embodiments, the second excipient comprises a positively charged group. In some embodiments, the second excipient comprises a permanently positively charged group. In some embodiments, the permanently positively charged group comprises a positive charge at a pH above 14. In some embodiments, the second excipient is distearoylphosphatidylcholine. In some embodiments, the pharmaceutical composition comprises an amount of the second excipient from about 0.5% w/w to about 20% w/w. In some embodiments, the amount of the second excipient is from about 2.5% w/w to about 15% w/w. In some embodiments, the amount of the second excipient is from about 5% w/w to about 10% w/w.

In some embodiments, the pharmaceutical composition further comprises a salt. In some embodiments, the salt is an ammonium salt. In some embodiments, the ammonium salt is ammonium acetate.

In some embodiments, the pharmaceutical composition comprises a ratio of clofazimine to amikacin sufficient to induce a synergistic antimicrobial effect against mycobacteria, wherein the synergistic effect is greater than the effect of either clofazimine to amikacin alone. In some embodiments, the synergistic effect is measured from the free base of amikacin. Similarly, the ratio of clofazimine to amikacin is measured based upon amikacin in its free base form. In some embodiments, the pharmaceutical composition comprises a ratio of clofazimine to amikacin is from about 5:1 to about 1:20. In some embodiments, the ratio of clofazimine to amikacin is from about 2:1 to about 1:15. In some embodiments, the ratio of clofazimine to amikacin is from about 1:1 to about 1:10. In some embodiments, the ratio of clofazimine to amikacin is 2:1, 1:1, 1:2, 1:4, or 1:8.

In some embodiments, the amikacin is an amikacin salt such as amikacin sulfate. In other embodiments, the amikacin is present in its free base form. In some embodiments, the pharmaceutical compositions have an X10 particle size distribution from about 0.25 μm to about 2.5 μm. In some embodiments, the X10 particle size distribution is from about 0.5 μm to about 2.0 μm. In some embodiments, the X10 particle size distribution is from about 1.0 μm to about 2.0 μm.

In some embodiments, the pharmaceutical compositions have an X50 particle size distribution from about 1.5 μm to about 5.0 μm. In some embodiments, the X50 particle size distribution is from about 2.0 μm to about 4.0 μm. In some embodiments, the X50 particle size distribution is from about 2.5 μm to about 3.5 μm.

In some embodiments, the pharmaceutical compositions have an X90 particle size distribution from about 2.5 μm to about 10.0 μm. In some embodiments, the X90 particle size distribution is from about 4.0 μm to about 9.0 μm. In some embodiments, the X90 particle size distribution is from about 5.0 μm to about 8.0 μm.

In some embodiments, the clofazimine and the amikacin have a difference in their median mass aerodynamic diameter (MMAD) of less than 5%. In some embodiments, the difference in MMAD is less than 3%. In some embodiments, the clofazimine and the amikacin have a difference in their fine particle fraction (FPF) of less than 5%. In some embodiments, the difference is measured using a device at a 2 kPa pressure. In some embodiments, the difference is measured using a device at a 4 kPa pressure. In some embodiments, the difference is measured at both a 2 kPa and a 4 kPa pressure.

In some embodiments, the clofazimine is deposited on the amikacin with a needle or rod morphology. In some embodiments, the pharmaceutical composition is a combined dose of clofazimine and amikacin, wherein the clofazimine is deposited on the surface of the amikacin and formulated for administration via inhalation. In some embodiments, the combined dose is from about 10 mg to about 50 mg. In some embodiments, less than 50% of the pharmaceutical composition is dissolved at pH 7 within 6 hours. In some embodiments, less than 25% of the pharmaceutical composition is dissolved. In some embodiments, less than 10% of the pharmaceutical composition is dissolved. In some embodiments, more than 25% of the pharmaceutical composition is dissolved at pH 4.5 within 24 hours. In some embodiments, more than 50% of the pharmaceutical composition is dissolved. In some embodiments, more than 75% of the pharmaceutical composition is dissolved.

In still yet aspect, the present disclosure provides methods of preparing a pharmaceutical composition comprising:
(A) obtaining a clofazimine solution containing clofazimine or a pharmaceutically acceptable salt thereof in a solvent;
(B) admix amikacin or a pharmaceutically acceptable salt thereof particles to the clofazimine solution to obtain a two-phase mixture;
(C) spray drying the two-phase mixture to obtain a pharmaceutical composition;
wherein the spray drying has a feed rate of greater than 10 mL/min.

In some embodiments, the amikacin is a solid. In some embodiments, the feed rate is greater than 25 mL/min. In some embodiments, the feed rate is greater than 40 mL/min. In some embodiments, the solvent is an alcohol. In some embodiments, the solvent is a C1-C4 alcohol such as ethanol or isopropanol.

In some embodiments, the clofazimine solution has a concentration of 0.1 mg/mL to about 5.0 mg/mL. In some embodiments, the concentration is from about 0.25 mg/mL to about 2.5 mg/mL. In some embodiments, the concentration is about 0.5 mg/mL. In some embodiments, the methods further comprise sonicating the clofazimine solution. In some embodiments, the amikacin is amikacin sulfate. In some embodiments, the particle size is reduced via homogenizing the two phase mixture. In some embodiments, the methods further comprise homogenizing the two-phase mixture. In some embodiments, the two-phase mixture is homogenized with a rotor stator homogenizer.

In some embodiments, the amikacin particles have been processed before to admixing. In some embodiments, the amikacin particles have been processed by spray drying. In some embodiments, the amikacin particles have been processed by jet milling.

In some embodiments, the amikacin particles have a X90 of less than 15 μm. In some embodiments, the amikacin particles have a X90 of less than 10 μm. In some embodiments, the amikacin particles have a X90 of less than 7.5 μm. In some embodiments, the two-phase mixture is generated by an anti-solvent precipitation. In some embodiments, the anti-solvent precipitation comprises dissolving the amikacin in water to form an aqueous amikacin solution. In some embodiments, the clofazimine is dissolved in an antisolvent to form a clofazimine antisolvent solution. In some embodiments, the antisolvent is an alcohol.

In some embodiments, the method comprises adding the aqueous amikacin solution to the clofazimine antisolvent solution. In some embodiments, the adding is done via nebulization. In some embodiments, the adding comprises adding a ratio of the aqueous amikacin solution volume to the clofazimine antisolvent solution from about 1:10 to about 1:250. In some embodiments, the ratio is from about 1:25 to about 1:150. In some embodiments, the ratio is from about 1:40 to about 1:75. In some embodiments, the amikacin particles are suspended in an antisolvent. In some embodiments, the amikacin particles have been prepared using jet milling. In some embodiments, the amikacin particles have been prepared using spray drying. In some embodiments, the amikacin particles are excipient free.

In some embodiments, admixing amikacin is carried out of over multiple steps. In some embodiments, the admixing amikacin comprises three discrete steps. In some embodiments, the admixing amikacin comprises a first step of adding amikacin to a first aliquot of clofazimine solution. In some embodiments, the method further comprises homogenization of the pharmaceutical composition, the clofazimine alone, or the amikacin alone to reduce particle size.

In some embodiments, the first aliquot is from about 1 mL to about 1 L. In some embodiments, the first aliquot is from about 5 mL to about 100 mL. In some embodiments, the first aliquot is about 25 mL. In some embodiments, the admixing amikacin comprises a second step of adding a second aliquot of clofazimine solution. In some embodiments, the second aliquot is in an amount sufficient to obtain a specific ratio of clofazimine and amikacin in the two-phase mixture.

In some embodiments, the specific ratio of clofazimine to amikacin is from about 5:1 to about 1:20. In some embodiments, the specific ratio of clofazimine to amikacin is from about 2:1 to about 1:15. In some embodiments, the specific ratio of clofazimine to amikacin is from about 1:1 to about 1:10. In some embodiments, the specific ratio of clofazimine to amikacin is 2:1, 1:1, 1:2, 1:4, or 1:8. In some embodiments, the admixing amikacin comprises a third step.

In some embodiments, the third step comprises adding additional solvent to obtain a final volume. In some embodiments, the additional solvent is the same as the solvent used in the clofazimine solution. In some embodiments, the additional solvent is a different solvent than the solvent used in the clofazimine solution. In some embodiments, the final volume is the capacity of the spray dryer apparatus.

In some embodiments, the methods further comprise using a spray dryer with an inlet temperature from about 50° C. to about 250° C. In some embodiments, the inlet temperature is from about 100° C. to about 200° C. In some embodiments, the inlet temperature is about 150° C. In some embodiments, the atomization air rate is set from about 1 to about 100. In some embodiments, the atomization air rate is from about 20 to about 60. In some embodiments, the atomization air rate is about 40. In some embodiments, the spray dryer comprises setting the atomizer such that the negative pressure is generated in the two-phase mixture. In some embodiments, the negative pressure is sufficient to pull the two-phase mixture into the spray dryer.

In still another aspect, the present disclosure provides pharmaceutical compositions prepared according to the methods described herein.

In another aspect, the present disclosure provides methods of treating or preventing a lung disease comprising administering to a patient in need thereof a therapeutically effective amount of a pharmaceutical composition described herein.

In some embodiments, the methods comprise treating the lung disease. In some embodiments, the methods comprise preventing the lung disease. In some embodiments, the lung disease is a lung infection such as an infection of a mycobacteria. In some embodiments, the mycobacteria are Mycobacterium abscessus, Mycobacterium avium, and Mycobacterium tuberculosis. In some embodiments, the mycobacteria are Mycobacterium abscessus. In other embodiments, the mycobacteria are Mycobacterium avium. In other embodiments, the mycobacteria are Mycobacterium tuberculosis.

In some embodiments, the methods further comprise administering another therapeutic agent. In some embodiments, the another therapeutic agent is another antibiotic such as another antibiotic that exhibits a synergistic or additive effect relative to the method comprising the pharmaceutical composition without the another antibiotic.

In some embodiments, the patient is a mammal such as a human. In some embodiments, the methods comprise administering the pharmaceutical composition once. In other embodiments, the methods comprise administering the pharmaceutical composition two or more times.

In yet another aspect, the present disclosure provides an inhaler comprising:
(A) a device capable of delivering a dose of one or more therapeutic agents;
(B) clofazimine; and
(C) amikacin
wherein the clofazimine and the amikacin are formulated to be delivered in the inhaler.

In some embodiments, the device is an inhaler. In some embodiments, the inhaler is a passive dry powder inhaler, a simple dry powder inhaler, a medium resistant dry powder, a capsule dosing chamber inhaler, or a dual dosing chamber inhaler. In some embodiments, the simple dry powder inhaler comprises less than 10 parts. In some embodiments, the medium resistant dry powder inhaler has a resistance of less than 0.025 kPa(½)/L/min. In some embodiments, the inhaler is a dual chamber inhaler. In some embodiments, the dual chamber inhaler comprises a first chamber with either clofazimine or amikacin and a second chamber with the other of clofazimine or amikacin.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows SEM images of the physically blended CFZ-AMK formulations.

FIG. 2 shows the physical blending of clofazimine and amikacin crystalline, air jet milled particles does not improve aerosol performance relative to the individual powders alone. In the case of amikacin, a decrease in aerosol performance was observed when the powder was physically blended with clofazimine, as indicated by the significant reduction in respirable fraction (RF<5 and RF<3)

FIG. 3 shows intracellular CFU of M. abscessus-infected THP-1 macrophages treated with CFZ-AMK physical mixtures. All treatments resulted in a significant decrease in CFU versus untreated control. High dose-100 μg/mL of powder administered; Low dose −20 μg/mL of powder administered.

FIGS. 4A-C shows excipient-free CFZ spray dried in ethanol at low feed flow rates (5 mL/min) results in the formation of elongated needle-shaped crystals. However, when spray dried in combination with AMK particles (B) at ultra-rapid feed flow rates (50 mL/min) the CFZ crystals are observed to precipitate onto the surface of the AMK particles (C). The needle/rod morphology is still observed; however, the AMK microparticle act as a scaffold to confine the growth of the crystals during the evaporation process.

FIG. 5 shows the incorporation of increasing amounts of clofazimine in the physical mixtures of clofazimine-amikacin increases the rate of intracellular killing. This is indicated by the increased change vs baseline control at earlier timepoints in the 1-0.5 PM formulation.

FIG. 6 shows excipient-free composite particles of CFZ and AMK can be prepared using an ultra-rapid spray drying approach. The ratio of CFZ-AMK may be adjusted by incorporating a higher concentration of AMK particles in the process.

FIG. 7 shows the apparatus configuration for anti-solvent precipitation method to produce two-phase clofazimine-amikacin sulfate feed stock. In this example, Drug A is amikacin sulfate, Drug B is clofazimine, Stabilizer 1 is polysorbate 80 and Stabilizer 2 is DSPC.

FIG. 8 shows the particle size distribution of two separate batches of amikacin microsuspensions produced by anti-solvent precipitation into a clofazimine solution (without high pressure homogenization).

FIG. 9 shows the particle size distribution of amikacin sulfate suspension prepared using anti-solvent precipitation before (circles) and after (squares) high pressure homogenization of the two-phase feed stock produced using anti-solvent precipitation.

FIG. 10 shows SEM images of the spray dried CFZ-AMK formulations.

FIG. 11 shows significant improvements in aerosol performance at 2 kPa and 4 kPa pressure drops are achieved with spray dried amikacin when prepared as a spray dried composite particle with clofazimine. This is indicated by the significant improvement in emitted fraction (EF) and respirable fraction (RF<5, RF<3) achieved when increasing amounts of clofazimine are present in the particles and compared to spray dried amikacin alone. Data presented as mean±SD (n=3); statistical significance determined using two-way ANOVA with Tukey post-hoc analysis.

FIG. 12 shows x-ray powder diffraction pattern of clofazimine-amikacin spray dried composite particles (CFZ-AMK-SD) indicates a partially crystalline material where clofazimine is in a crystalline phase (based upon the observed peaks corresponding to crystalline unprocessed clofazimine) and amikacin is in an amorphous phase (based upon the lack of observed peaks corresponding to crystalline unprocessed amikacin.

FIG. 13 shows intracellular CFU of M. abscessus-infected THP-1 macrophages treated with CFZ-AMK spray dried composite particles. All treatments resulted in a significant decrease in CFU versus untreated control.

FIG. 14 shows intracellular CFU of M. abscessus-infected THP-1 macrophages treated with CFZ-AMK spray dried composite particles or physical mixtures for 72 hours.

FIG. 15 shows the increased intracellular antibacterial activity is observed with the composite CFZ-AMK-SD particles compared to micronized amikacin (AMK-M) or micronized clofazimine (CFZ-M) alone, though this effect was decreased in the formulation which had the lowest level of clofazimine (CFZ-AMK-SD 1-8).

FIG. 16 shows the statistically significant improvements in intracellular and extracellular antibacterial activity are achieved with composite spray dried CFZ-AMK particles compared to physically blended clofazimine-amikacin powder at several different ratios. Data presented as mean±SD (n=3). Statistically analysis was assessed using two way ANOVA with Tukey's multiple comparisons test.

### DESCRIPTION OF ILLUSTRATIVE EMBODIMENTS

The composition comprises of composite particles containing clofazimine and amikacin (referred to as CFZ-AMK herein) which are delivered to the airways via oral powder inhalation for the treatment of mycobacterial lung infections. The composite particles are engineered in such a way that the stabilizing or flow-enhancing excipients are not required thereby reducing the inhalation burden, and a synergistic pharmacological effect is induced against intracellular mycobacterial populations.

Through independent in vitro evaluation of the anti-mycobacterial activity of CFZ and AMK, several ratios that exhibit synergistic activity were identified (FIG. 8A). Additionally, an in vitro efficacy study utilizing Mab-infected THP-1 macrophages revealed that the addition of micronized CFZ particles to cell culture media containing 40 μg/mL AMK resulted in a statistically significant reduction in intracellular Mab bacilli compared to the use of AMK alone (FIG. 8B). Interestingly, no statistically significant changes in intracellular bacterial burden were noted with increasing doses of micronized CFZ, despite a notable increase in intracellular drug accumulation. These results indicate that potentially only a portion of the intracellular CFZ is available to interact with Mab bacilli, and that complete sterilization requires the intracellular accumulation of a synergistic antibiotic, such as AMK.

Also provided herein are methods of preparing and using these compositions containing amikacin and clofazimine. Details of these compositions are provided in more detail below.

### I. Pharmaceutical Compositions

In some aspects, the present disclosure provides pharmaceutical compositions comprising amikacin and clofazimine that may be formulated for administration to the lungs, such as via inhalation. In some embodiments, the clofazimine is deposited on the amikacin with a needle or rod morphology. The deposited clofazimine is deposited on the amikacin with an aspect ratio from about 0.75 to 1, or no less than 0.75. In some embodiments, the pharmaceutical composition is a combined dose of clofazimine and amikacin, wherein the clofazimine is deposited on the surface of the amikacin and formulated for administration via inhalation. In some embodiments, the clofazimine is deposited on the surface of the amikacin.

In particular, the pharmaceutical composition comprises a mixture of clofazimine and amikacin. Generally, the ratio of clofazimine to amikacin in the mixture may be sufficient to induce a synergistic antimicrobial effect against mycobacteria. For example, the mixture may have a ratio of clofazimine to amikacin from about 5:1 to about 1:20, from about 2:1 to about 1:15, from about 1:1 to about 1:10, or from about 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, to about 1:20, or any range derivable therein.

In some embodiments, pharmaceutical composition has an X10 particle size distribution from about 0.25 μm to about 2.5 μm, from about 0.5 μm to about 2.0 μm, from about 1.0 μm to about 2.0 μm, or from about 0.25 μm, 0.50 μm, μm, 0.75 μm, 1.0 μm, 1.25 μm, 1.50 μm, 1.75 μm, 2.0 μm, 2.25 μm, to about 2.5 μm, or any range derivable therein. In some embodiments, pharmaceutical composition has an X50 particle size distribution from about 1.5 μm to about 5.0 μm, from about 2.0 μm to about 4.0 μm, from about 2.5 μm to about 3.5 μm, or from about 1.5 μm, 2.0 μm, 2.5 μm, 3.0 μm, 3.5 μm, 4.0 μm, 4.5 μm, to about 5.0 μm, or any range derivable therein. In some embodiments, pharmaceutical composition has an X90 particle size distribution from about 2.5 μm to about 10.0 μm, from about 4.0 μm to about 9.0 μm, from about 5.0 μm to about 8.0 μm, or from about 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, to about 10.0 μm, or any range derivable therein. In some embodiments, the pharmaceutical composition may be less than 50%, less than 40%, less than 30%, less than 25%, less than 20%, less than 15%, or less than 10% dissolved at a pH of about 7. In some embodiments, the pH is physiological pH. In some embodiments, the pharmaceutical composition may be more than 10%, 15%, 20%, 25%, 30%, 35%, 40%, 45%, 50%, 55%, 60%, or 75% dissolved at an acid pH such as at pH 4.5 within 24 hours.

### A. Clofazimine

The pharmaceutical compositions described herein comprise clofazimine as an active agent. As used herein, the term “clofazimine” or “CFZ” refers to N,5-bis(4-chlorophenyl)-3-(1-methylethylimino)-5H-phenazin-2-amine in any of its forms, including non-salt and salt forms (e.g., clofazimine mesylate), esters, anhydrous and hydrate forms of non-salt and salt forms, solvates of non-salt and salts forms, its enantiomers (R and S forms, which may also by identified as d and 1 forms), and mixtures of these enantiomers (e.g., racemic mixture, or mixtures enriched in one of the enantiomers relative to the other). In some embodiments, less than 25%, less than 10%, less than 5%, less than 2%, or less than 1% of the clofazimine is present as elongated needle-crystals. The clofazimine crystals maybe deposited on amikacin particles. These amikacin particles are, in some embodiments, spherical in nature.

In the preparation of compositions with clofazimine, solubility is a major limiting factor to the development of a pharmaceutically acceptable formulation of clofazimine. Clofazimine is practically insoluble in water. Additionally, this highly beneficial antibiotic exhibits limited solubility in a variety of other solvents. According to the Merck Index, clofazimine is soluble in DMF and benzene, soluble in 15 parts chloroform, 700 parts ethanol, 1000 parts ether, sparingly soluble in acetone and ethyl acetate and practically insoluble in water. It has also been reported that a 0.1% clofazimine solution in methanol can be formed (Sabnis et al., 2015). The International Council for Harmonization of Technical Requirements for Pharmaceuticals for Human Use (ICH) guidance for industry Q3C Impurities: Residual Solvents recognizes benzene as a Class 1 solvent (should not be employed in the manufacture of drug products; 2 ppm concentration limit), chloroform, methanol, acetonitrile and are Class 2 solvents (should be limited in drug products due to inherent toxicity; 60 ppm, 3000 ppm, and 410 ppm, respectively), and dilute acetic acid and ethanol are listed as recognized as Class 3 solvents. Considering both the large volumes required for full dissolution as well as the safety limitations of the use of these solvents, manufacturing of respirable clofazimine particles via commonly used constructive (bottom-up) particle engineering techniques for dry powder formulation such as spray drying is extremely challenging. Successful preparation of respirable clofazimine particles is reported to require addition of excipients to the formulation, such as leucine or dipalmitoylphosphatidylcholine (DPPC), in order to formulate a product suitable for lung deposition (1, 2). Spray drying of pure clofazimine in organic solvents such as ethanol or methanol results in formation of poorly dispersible needle-shaped crystals. If a supersaturated solution of clofazimine is formulated for the liquid feed, a multimodel size distribution results, potentially due to the drug precipitating out of the liquid feed prior to complete drying of the droplets. If a saturated solution is formulated for the organic solvent feed, defined as complete dissolution of clofazimine in the solvent, a partially amorphous formulation of clofazimine results, which is prone to physicochemical instability. Thus, methods to prepare clofazimine compositions with other drugs are of importance.

### B. Amikacin

The pharmaceutical compositions described herein comprise amikacin as an active agent. Amikcain is an antibiotic that is used to treat bacterial infections derived from kanamycin. This compound is an aminoglycoside antibiotic and related to other aminoglycoside antibiotics. These compounds useful in treating a wide variety of different bacterial infections include mycobacteria infections. This antibiotic is known to target the 16S rRNA and the RNA binding S12 protein thereby inhibiting protein synthesis. Generally, amikacin is known for its ability to escape the vast majority of bacteria resistance mechanism except mutations in 16S rRNA and in the proteins acetyltransferases and adenylyltransferases. Aminoglycosides are known to cause kidney damage as well as ototoxicity in 1-10% of uses. Furthermore, these side effects are increased in cases where long term and high doses are utilized. Additionally, the liposomal formulations which have previously been used as a inhalable suspension of amikacin are known to trigger a wide variety of different respiratory side effects such as pneumonitis, bronchospasms, and hemoptysis in addition to other common side effects such as coughing, airway irritation, pain, fatigue, diarrhea, nausea, or difficulty speaking. In the context of this application, the term “amikacin” refers to (2S)-4-amino-N-[(2S,3S,4R,5S)-5-amino-2-[(2S,3R,4S,5S,6R)-4-amino-3,5-dihydroxy-6-(hydroxymethyl)oxan-2-yl]oxy-4-[(2R,3R,4S,5R,6R)-6-(aminomethyl)-3,4,5-trihydroxy-oxan-2-yl]oxy-3-hydroxy-cyclohexyl]-2-hydroxybutanamide in any of its forms, including non-salt and salt forms (e.g., amikacin sulfate), esters, anhydrous and hydrate forms of non-salt and salt forms, solvates of non-salt and salts forms, its enantiomers (R and S forms, which may also by identified as d and l forms), and mixtures of these enantiomers (e.g., racemic mixture, or mixtures enriched in one of the enantiomers relative to the other).

### C. Excipients

In some aspects, the present disclosure comprises one or more excipients formulated into pharmaceutical compositions. An “excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Furthermore, these compounds may be used as diluents in order to obtain a dosage that can be readily measured or administered to a patient. Non-limiting examples of excipients include stabilizing agents, surfactants, surface modifiers, solubility enhancers, buffers, encapsulating agents, antioxidants, preservatives, nonionic wetting or clarifying agents, viscosity increasing agents, and absorption-enhancing agents.

The pharmaceutical composition may comprise an excipient. When an excipient is present in the composition, the amount of the excipient in the composition is from about 0.00001% to about 70% w/w, from about 0.001% to about 40% w/w, from about 0.01% to about 30% w/w, or from about 0.1% to about 20% w/w of the total weight of the pharmaceutical composition. In some embodiments amount of the excipient in the pharmaceutical composition comprises from about 0.001%, 0.005%, 0.01%, 0.05%, 0.1%, 0.125%, 0.15%, 0.2%, to about 0.25% w/w, or any range derivable therein, of the total pharmaceutical composition. In some embodiments, the amount of the excipient in the pharmaceutical composition is at 0.05% to 0.25% w/w of the total weight of the pharmaceutical composition. Alternatively, the amount of the excipient in the pharmaceutical composition comprises from about 1%, 5%, 10%, 15%, 20%, 25%, 30%, 35%, 40%, 45%, 50%, 55%, 60%, 70%, to about 80% w/w, or any range derivable therein, of the total pharmaceutical composition. In some embodiments, the amount of the excipient in the pharmaceutical composition is at 20% to 40% w/w of the total weight of the pharmaceutical composition. In some embodiments, the pharmaceutical composition is formulated without any excipients.

### D. Uses of Compositions

In some aspects, the present compositions may be used to treat an infection of a mycobacterium such as Mycobacterium abscessus (Mab), Mycobacterium avium complex, and Mycobacterium tuberculosis complex. The infection may be, but is not limited to, Mycobacterium tuberculosis, multi-drug resistant M. tuberculosis, extensively drug resistant M. tuberculosis, Mycobacterium avium complex, Mycobacterium abscesses, Mycobacterium kansasii, Staphylococcus aureus, and methicillin resistant Staphylococcus aureus (MRSA). These compositions may show an enhanced effect in treating infections with a combination of two or more active agents. These active agents may result in increased treatment options for these infections. In particular, compositions may show an enhanced such as additive or synergistic effect in treating the infection. In some embodiments, the treatment may be prophylactic to subjects at risk of developing a pulmonary infection, such as subjects with a family member diagnosed with a pulmonary infection, subjects traveling to areas with high rates of pulmonary infection, or healthcare workers.

In some embodiments, treatment of a patient with the present pharmaceutical compositions may comprise modulated drug release. In some embodiments, the pharmaceutical composition may be formulated for slow- or delayed-release. In some embodiments, the pharmaceutical composition may be formulated for fast-release. In further embodiments, the pharmaceutical composition may be formulated for both slow and fast release (i.e., dual release profile).

In some embodiments, the pharmaceutical composition may be administered on a routine schedule. As used herein, a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In some embodiments, the pharmaceutical composition is administered once per day. In preferred embodiments, the pharmaceutical composition is administered less than once per day, such as every other day, every third day, or once per week. In some embodiments, a complete dose of the pharmaceutical composition is between 1-100 mg, such as 20-100, 50-100, 10-20, 20-40, 50-70, or 80-90 mg.

In some embodiments, the dissolution rate of the pharmaceutical composition is measured. In some embodiments, crystalline clofazimine has a slow dissolution rate. In some embodiments, the dissolution rate of clofazimine is such that no more than 30%, such as less than 25, 20, 15, or 10%, of the clofazimine by mass dissolves in dissolution media within 15 minutes of addition. In some embodiments, the dissolution media is Phosphate Buffered Saline pH 7.4+0.2% polysorbate 80.

In some embodiments, clofazimine is internalized by J774.A1 macrophage cultures. In some embodiments, the clofazimine is crystalline. In some embodiments the clofazimine is micronized. In some embodiments, micronized crystalline clofazimine particles are internalized by J774.A1 macrophage cultures. In further embodiments, the rate of internalization of the particles by macrophages is high, such as greater than 80% internalization after 8 hours of incubation. In some embodiments, macrophages transform the clofazimine into a different crystalline-like form. In some embodiments, change in crystalline form of clofazimine is detected by a fluorescence shift. In some embodiments, the fluorescence shift is from around 590 nm to around 660 nm. In some embodiments, the fluorescence shift occurs within a short time. In some embodiments, the fluorescence shift occurs within 1 week, such as in 7 days, 6 days, 5 days, 4, days 3 days, 2 days, or within 24 hours.

In some embodiments, the treatment methods provided herein may further comprise administering at least a second therapeutic agent. The second agent may be, but is not limited to, bedaquilline, pyrazinamide, nucleic acid inhibitors, protein synthesis inhibitors, and cell envelope inhibitors. The group protein synthesis inhibitors may include, but are not limited to, linezolid, clarithromycin, amikacin, kanamycin, capreomycin, and streptomycin. The group cell envelope inhibitors may include, but are not limited to, ethambutol, ethionamide, thioacetizone, isoniazid, imipenem, clavulanate, cycloserine, terizidone, amoxicillin, and prothionamide. The group nucleic acid inhibitors may include, but are not limited to, rifampicin, rifabutin, rifapentine, 4-aminosalicylic acid, moxifloxacin, ofloxacin, and levofloxacin. In some embodiments, the second therapeutic agent may be clofazimine. Other exemplary agents include but are not limited to vancomycin, tobramycin, ciprofloxacin, fosfomycin, and rifaximin. The combination therapies may be administered simultaneously, sequentially, or separately.

### E. Inhalation

In some embodiments, the present disclosure relates to respirable particles must be in the aerodynamic size range, such as mean median aerodynamic diameter of around 2 to 10 microns or 4 to 8 microns in aerodynamic diameter. In some embodiments, the present disclosure provides methods for the administration of the inhalable niclosamide composition provided herein using a device. Administration may be, but is not limited, to inhalation of niclosamide using an inhaler. In some embodiments, an inhaler is a simple passive dry powder inhaler (DPI), such as a Plastiape RSO1 monodose DPI. In a simple dry powder inhaler, dry powder is stored in a capsule or reservoir and is delivered to the lungs by inhalation without the use of propellants.

In some embodiments, an inhaler is a single use, disposable inhaler such as a single-dose DPI, such as a DoseOne™, Spinhaler, Rotohaler®, Aerolizer®, or Handihaler. These dry powder inhaler may be a passive DPI. In some embodiments, an inhaler is a multidose DPI, such as a Plastiape RS02, Turbuhaler®, Twisthaler™, Diskhaler®, Diskus®, or Ellipta™. In some embodiments, the inhaler is Twincer®, Orbital®, TwinCaps®, Powdair, Cipla Rotahaler, DP Haler, Revolizer, Multi-haler, Twister, Starhaler, or Flexhaler®. In some embodiments, an inhaler is a plurimonodose DPI for the concurrent delivery of single doses of multiple medications, such as a Plastiape RS04 plurimonodose DPI. Dry powder inhalers have medication stored in an internal reservoir, and medication is delivered by inhalation with or without the use of propellants. Dry powder inhalers may require an inspiratory flow rate greater than 30 L/min for effective delivery, such as between about 30-120 L/min.

In some embodiments, the inhalable pharmaceutical composition is delivered as a propellant formulation, such as HFA propellants.

In some embodiments, the inhaler may be a metered dose inhaler. Metered dose inhalers deliver a defined amount of medication to the lungs in a short burst of aerosolized medicine aided by the use of propellants. Metered dose inhalers comprise three major parts: a canister, a metering valve, and an actuator. The medication formulation, including propellants and any required excipients, are stored in the canister. The metering valve allows a defined quantity of the medication formulation to be dispensed. The actuator of the metered dose inhaler, or mouthpiece, contains the mating discharge nozzle and typically includes a dust cap to prevent contamination.

In some embodiments, an inhaler is a nebulizer or a soft-mist inhaler such as those described in WO 1991/14468 and WO 1997/12687, which are incorporated herein by reference. A nebulizer is used to deliver medication in the form of an aerosolized mist inhaled into the lungs. The medication formulation be aerosolized by compressed gas, or by ultrasonic waves. A jet nebulizer is connected to a compressor. The compressor emits compressed gas through a liquid medication formulation at a high velocity, causing the medication formulation to aerosolize. Aerosolized medication is then inhaled by the patient. An ultrasonic wave nebulizer generates a high frequency ultrasonic wave, causing the vibration of an internal element in contact with a liquid reservoir of the medication formulation, which causes the medication formulation to aerosolize. Aerosolized medication is then inhaled by the patient. In some embodiments, the single use, disposable nebulizer may be used herein. A nebulizer may utilize a flow rate of between about 3-12 L/min, such as about 6 L/min. In some embodiments, the nebulizer is a dry powder nebulizer.

In some embodiments, the pharmaceutical composition may be provided in a unit dosage form, such as in a capsule, blister or a cartridge, wherein the unit dose comprises at least 10 mg of the pharmaceutical composition, such as at least 15 mg or 20 mg of the pharmaceutical composition per dose. In particular aspects, the unit dosage form does not comprise the administration or addition of any excipient and is merely used to hold the powder for inhalation (i.e., the capsule, blister, or cartridge is not administered). In some embodiments, the pharmaceutical composition may be administered in a high emitted dose, such as at least mg, preferably at least 15 mg, even more preferably 20 mg. In some embodiments, administration of the pharmaceutical composition results in a high fine particle dose into the deep lung such as greater than 5 mg. Preferably, the fine particle dose into the deep lung is at least 10 mg, even more preferably at least 15 mg. In some aspects, the fine particle dose is at least, 50%, such as at least 60, 65, 70, 75, or 80% of the emitted dose.

In some embodiments, changes in pressure drop across the device result in a change in emitted dose. In some embodiments, changes in pressure drop across the device of 3 kPa, such as from 4 kPa to 1 kPa, result in a reduction of emitted dose of less than 25%, such as 24%, 23%, 22%, 21%, 20%, 19%, 18%, 17%, 16%, 15%, 14%, 13%, 12%, 11%, 10%, 9%, 8%, 7%, 6%, 5% or less. In some embodiments, changes in inhalation pressure drop across the device result in a change in fine particle dose. In some embodiments, changes in inhalation pressure drop across the device of 3 kPa, such as from 4 kPa to 1 kPa result in a reduction of fine particle dose of less than 15%, such as 14%, 13%, 12%, 11%, 10%, 9%, 8%, 7%, 6%, 5% or less.

### II. Manufacturing Methods

In some aspects, the present disclosure provides methods of preparing a pharmaceutical composition comprising: (A) obtaining a clofazimine solution containing clofazimine in a solvent; (B) admixing amikacin to the clofazimine solution to obtain a two-phase mixture; (C) spray drying the two-phase mixture to obtain a pharmaceutical composition; wherein the spray drying has a feed rate of greater than 10 mL/min.

In some embodiments, the solvent is an alcohol. In further embodiments, the solvent is a C1-C4 alcohol, such as ethanol or isopropanol. In some embodiments, the solution has a concentration of 0.1 mg/mL to about 5.0 mg/mL, from about 0.25 mg/mL to about 2.5 mg/mL, or from about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 0.10 mg/mL, 0.15 mg/mL, 0.20 mg/mL, 0.25 mg/mL, 0.5 mg/mL, 0.75 mg/mL, 1.0 mg/mL, 1.5 mg/mL, 2.0 mg/mL, 2.5 mg/mL, 3.0 mg/mL, 3.5 mg/mL, 4.0 mg/mL, 4.5 mg/mL, to about 5.0 mg/mL, or any range derivable therein.

In some embodiments, the methods further comprise sonicating the clofazimine solution. In some embodiments, the amikacin is amikacin sulfate. In some embodiments, the methods further comprise homogenizing the two-phase mixture. In some embodiments, the two-phase mixture is homogenized with a rotor stator homogenizer.

In some embodiments, admixing amikacin is carried out of over multiple steps. In further embodiments, admixing amikacin comprises three discrete steps. In some embodiments, admixing amikacin comprises a first step of adding amikacin to a first aliquot of clofazimine solution. In some embodiments, the first aliquot is from about 1 mL to about 1 L, from about 5 mL to about 100 mL, or from about 1 mL, 2 mL, 3 mL, 4 mL, 5 mL, 6 mL, 7 mL, 8 mL, 9 mL, 10 mL, 15 mL, 20 mL, 25 mL, 30 mL, 35 mL, 40 mL, 45 mL, 50 mL, 100 mL, 150 mL, 200 mL, 250 mL, 500 mL, 750 mL, to about 1 L, or any range derivable therein.

In some embodiments, admixing amikacin comprises a second step of adding a second aliquot of clofazimine solution. In some embodiments, the second aliquot is an amount sufficient to obtain a specific ratio of clofazimine and amikacin in the two-phase mixture. In some embodiments, the specific ratio of clofazimine to amikacin is from about 5:1 to about 1:20, from about 2:1 to about 1:15, from about 1:1 to about 1:10, or from about 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:8, 1:19, to about 1:20, or any range derivable therein.

In some embodiments, admixing amikacin comprises a third step. In some embodiments, the third step comprises adding additional solvent to obtain a final volume. In some embodiments, the additional solvent is the same as the solvent used in the clofazimine solution. In other embodiments, the additional solvent is a different solvent than the solvent used in the clofazimine solution. In some embodiments, the final volume is the capacity of the spray dryer apparatus.

### A. Spray Drying

Thus, the final formulations may be prepared using a spray drying technique. Spray drying is a process that converts a liquid feed to a dried particulate form. The process may further comprise a secondary drying process, such as fluidized bed drying or vacuum drying, may be used to reduce residual solvents to pharmaceutically acceptable levels. Typically, spray-drying involves contacting a highly dispersed liquid suspension or solution with a sufficient volume of hot air or other gas to produce evaporation and drying of the liquid droplets. In a standard procedure, the composition is sprayed into a current of warm filtered air or gas that evaporates the solvent and conveys the dried product to a collector. The spent air is then exhausted with the solvent, or alternatively the spent air is sent to a condenser to capture and potentially recycle the solvent. The spray is emitted through a nozzle such as a pressure nozzle, a two-fluid electrosonic nozzle, a two-fluid nozzle, or a rotary atomizer. Commercially available types of apparatus may be used to conduct the spray-drying such as those manufactured by Buchi Ltd. and Niro, or described in US 2004/0105820 and US 2003/0144257.

Spray-drying typically employs a solids loading of material from about 0.25% to about 30% such as about 1% solids loading. If the solids loading is too low, then the composition may be unable to be formulated commercially or result in a product that is too dilute to be useful. On the other hand, the upper limit of solids loading is governed by the viscosity of the resulting solution and the solubility of the components in the solution. This material may be fed from the spray dryer at a feed flow rate greater than about 10 mL/min, 15 mL/min, 20 mL/min, 25 mL/min. 30 mL/min, 35 mL/min, 40 mL/min, 45 mL/min, 50 mL/min, 55 ml/min, 60 mL/min, 65 mL/min, 70 mL/min. 75 ml/min, 80 mL/min, 85 mL/min, 90 mL/min. 95 mL/min, or 100 mL/min.

Techniques and methods for spray-drying may be found in Perry's Chemical Engineering Handbook. 6th Ed., R. H. Perry, D. W. Green & J. O. Maloney, eds.), McGraw-Hill book co. (1984); and Marshall “Atomization and Spray-Drying” 50, Chem. Eng. Prog. Monogr. Series 2 (1954). In general, the spray-drying is conducted with an inlet temperature of from about 40° C. to about 200° C., for example, from about 50° C. to about 250° C., or from about 100° C. to about 200° C. The inlet temperature may from about 35° C., 40° C. 50° C., 60° C., 70° C., 80° C., 90° C., 100° C., 110° C., 120° C., 130° C., 140° C., 150° C., 160° C., 170° C., 180° C., 200° C., 220° C., 225° C., 240° C., to about 250° C.′, or any range derivable therein.

In some embodiments, the atomization air rate is from about 20 to about 60, or from about 20, 25, 30, 35, 40, 45, 50, 55, to about 60, or any range derivable therein. In some embodiments, the spray dryer comprises setting the atomizer such that the negative pressure is generated in the two-phase mixture. In some embodiments, the negative pressure is sufficient to pull the two-phase mixture into the spray dryer.

### III. Definitions

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified polysaccharide, wherein at least one of the monomeric saccharide units is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

The term “dissolution” as used herein refers to a process by which a solid substance, here the active ingredients, is dispersed in molecular form in a medium. The dissolution rate of the active ingredients of the pharmaceutical dose of the invention is defined by the amount of drug substance that goes in solution per unit time under standardized conditions of liquid/solid interface, temperature and solvent composition.

The term “deposition” or deposit” refers to the growth a pharmaceutical composition by the placement of one or more molecules of a compound or composition on a surface.

As used herein, the term “aerosols” refers to dispersions in air of solid or liquid particles, of fine enough particle size and consequent low settling velocities to have relative airborne stability (See Knight, V., Viral and Mycoplasmal Infections of the Respiratory Tract. 1973, Lea and Febiger, Phila. Pa., pp. 2).

As used herein, the term “physiological pH” refers to a solution with is at its normal pH in the average human. In most situation, the solution has a pH of approximately 7.4.

As used herein, “inhalation” or “pulmonary inhalation” is used to refer to administration of pharmaceutical preparations by inhalation so that they reach the lungs and in particular embodiments the alveolar regions of the lung. Typically inhalation is through the mouth, but in alternative embodiments in can entail inhalation through the nose.

As used herein, “dry powder” refers to a fine particulate composition that is not suspended or dissolved in an aqueous liquid.

A “simple dry powder inhaler” refers a device for the delivery of medication to the respiratory tract, in which the medication is delivered as a dry powder in a single-use, single-dose manner. In particular aspects, a simple dry powder inhaler has fewer than 10 working parts. In some aspects, the simple dry powder inhaler is a passive inhaler such that the dispersion energy is provided by the patient's inhalation force rather than through the application of an external energy source.

A “median particle diameter” refers to the geometric diameter as measured by laser diffraction or image analysis. In some aspects, at least either 50% or 80% of the particles by volume are in the median particle diameter range.

A “Mass Median Aerodynamic Diameter (MMAD)” refers to the aerodynamic diameter (different than the geometric diameter) and is measured by laser diffraction.

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±5% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “essentially free of” or “essentially free” is used to represent that the composition contains less than 1% of the specific component. The term “entirely free of” or “entirely free” contains less than 0.1% of the specific component.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. Examples

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1-Generation of Physical Blends of Clofazimine and Amikacin

### A. Manufacturing of Powders

As a simple and cost-effective approach to the generation of a composite CFZ-AMK particle using advanced particle engineering techniques, physical blending of micronized CFZ and micronized amikacin sulfate (AMK-S) was assessed. Using a Model 00 Jet-O-Mizer Air Jet Mill (fluid energy), AMK-S was micronized to a X50 particle diameter of 4.04 μm and an X90 particle diameter of 7.72 μm, and CFZ was micronized to a X50 particle diameter of 2.39 μm and an X90 particle diameter of 5.29 μm. The powders were combined to generate the following ratios (AMK weight was adjusted to account for the 75% assay in amikacin sulfate): 1:0.5 CFZ-AMK, 1:1 CFZ:AMK, 1:2 CFZ-AMK, 1:4 CFZ:AMK, and 1:8 CFZ:AMK. The powders were initially mixed in a mortar and pestle using a process of geometric dilution, and then mixed for 1 hour using a Turbula powder blender. In separate experiment, uniform physical blends were generated using a mortar and pestle using geometric dilution.

The content uniformity of the clofazimine component of the powder was assessed, with a target % CV of less than 5. This was performed by dissolving a known amount of the physically blended powder in a 75-25 mixture of acetonitrile and water containing 0.13 mg/mL polysorbate 80, and adding a volume of 5 M ammonium acetate equivalent to 16.6% of the original solution volume to induce phase separation. The upper organic phase was assayed for clofazimine using UV absorbance (285 nm) and concentration was quantified by using a standard curve of CFZ spiked concentrations (which had undergone the same phase separation procedure). The results of the content uniformity assay are described in Tables 1 and 2 below.

TABLE 1
Content Uniformity of CFZ-AMK physical
mixtures prepared using powder blender

| Ratio | Average CFZ content | Coefficient of variation |
|---|---|---|
| (CFZ:AMK) | (%) | (%) |
| 1:1 | 40.9 | 1.5 |
| 1:4 | 16.7 | 4.3 |
| 1:8 | 8.7 | 2.9 |

TABLE 2
Content Uniformity of CFZ-AMK physical
mixtures prepared using mortar and pestle

| Ratio | Average CFZ content | Coefficient of variation |
|---|---|---|
| (CFZ:AMK) | (%) | (%) |
| 1:0.5 | 57.7 | 1.0 |
| 1:2 | 25.2 | 1.5 |
| 1:4 | 13.4 | 3.7 |
| 1:8 | 7.0 | 4.0 |

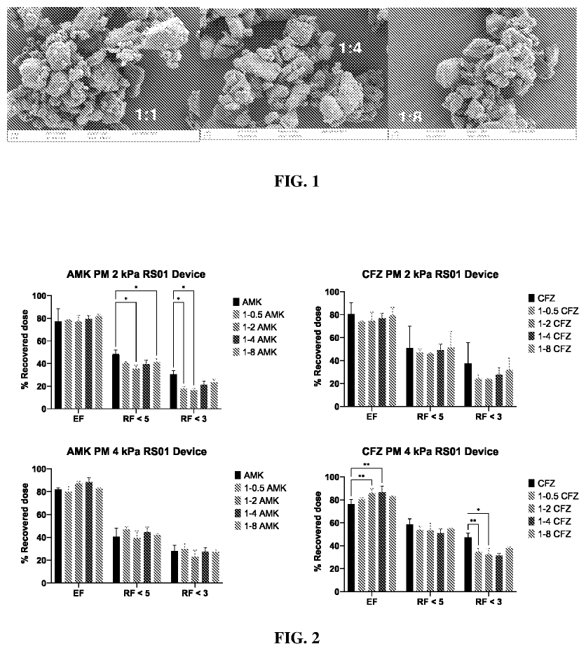

The resulting particle size distribution (PSD) of the physical mixtures are described in Table 3 and 4, while FIG. 1 shows the particle morphology of the formulations. PSD was calculated using Fraunhofer theory using the Sympatec laser diffractor. Geometric particle size distribution of the spray dried powder was assessed with a HELOS laser diffraction instrument (Sympatec GmbH, Germany) using RODOS dry dispersion at 3 bar dispersion pressure, 20% feed line rotation. Measurements were taken every 5 msec 10 following powder dispersion. Measurements that were between 5-25% optical density were averaged to determine particle size distribution. Data was analyzed using HELOS software version 5.6.0.0 with an HRLD model to convert the scattering pattern to a particle size distribution.

TABLE 3
Particle size distribution of CFZ-AMK physical
mixtures prepared using powder blender

| Sample | X10 (μm) | X50 (μm) | X90 (μm) |
|---|---|---|---|
| 1:1 PM | 1.16 | 3.38 | 7.30 |
| 1:4 PM | 1.29 | 3.84 | 7.86 |
| 1:8 PM | 1.34 | 3.91 | 8.03 |

TABLE 4
Particle size distribution of CFZ-AMK physical
mixtures prepared using mortar and pestle

| Sample | X10 (μm) | X50 (μm) | X90 (μm) |
|---|---|---|---|
| 1:0.5 PM | 0.85 | 2.59 | 6.05 |
| 1:2 PM | 1.07 | 3.65 | 7.42 |
| 1:4 PM | 1.01 | 3.45 | 7.03 |
| 1:8 PM | 1.01 | 3.48 | 7.06 |

### B. Aerosol Performance of Physically Blended CFZ and AMK

The aerosol performance of the physically blended mixtures was determined using cascade impaction, specifically as Next Generation Impactor, as prescribed by the United States Pharmacopeia chapter 601. For these studies, a medium resistance Model 7 RS01 dry powder inhaler (DPI) manufactured by Berry Global Osnago was utilized. 20 mg of the physical blends (1:0.5 CFZ-AMK, 1:2 CFZ-AMK, 1:4 CFZ-AMK, or 1:8 CFZ-AMK) or 10 mg of air jet milled clofazimine or air jet milled amikacin sulfate alone was loaded into a size 3 hydroxypropyl methylcellulose (HPMC) capsule. The device was actuated at an airflow rate to induce a 4 kPa pressure drop (i.e., 80 L/min) or 2 kPa pressure drop (i.e., 56.6 L/min) through the device for period of time sufficient to draw 4 L of air through the apparatus. To reduce particle bounce and re-entrainment, the NGI plates were coated with 1% (v/v) glycerin in ethanol and allowed to dry prior to performing the experiment. After actuation, powder was collected from the capsule, device, induction port, and stages of the Next Generation Impactor by adding a volume of 75/25 acetonitrile/water containing 0.16 mg/mL polysorbate 80 that was sufficient to dissolve the deposited powder. Phase separation of the solution into an organic phase containing clofazimine and an aqueous phase containing amikacin was induced by adding a volume of 5 M ammonium acetate equivalent to 16.7% of the original solution volume. The organic and aqueous phases were analyzed using high pressure liquid chromatography and concentration of each drug was quantified using a previously prepared standard curve.

Stage 1-7 cut-off diameters were determined using equation 1 and MOC cut-off diameters were determined using equation 2.

$${D}_{50 , Q} = {{D}_{50 , Qn} ( \frac{{Q}_{n}}{Q} )}^{X} \qquad Eq ( 1 )$$

$${D}_{80 , Q} = 0.14 {( \frac{{Q}_{n}}{Q} )}^{1.36} \qquad Eq ( 2 )$$

where D_{50,Q} is the cutoff diameter at the flow rate, Q, and the subscript, n, refers to the archival reference value for Qₙ=60 L/min, and the values for the exponent, x, were determined by the archival NGI stage cut size-flow rate calculations, as determined by Marple et al.

The emitted fraction (EF) was calculated as the total drug emitted from the device as a percentage of the total mass of drug collected (i.e., recovered dose). The respirable fraction that was less than 5 μm aerodynamic diameter and less than 3 μm aerodynamic diameter corresponded to the percentage of the recovered dose predicted to have the aerodynamic diameter below 5 μm and 3 μm. The respirable fraction values were interpolated from a graph with the cumulative percentage of the emitted dose deposited downstream from an NGI stage as the ordinate and the particle cutoff size of that stage as the abscissa. For each sample, the mass median aerodynamic diameter (MMAD), which represents the mass-based median point of the aerodynamic particle size distribution (APSD), and geometric standard deviation (GSD), which represents the spread of the APSD, were determined by plotting the cumulative percentage of mass less than the stated aerodynamic size cut (expressed as Probits) against the aerodynamic diameter (log scale). Distributions were log normal. A linear regression was performed to determine the aerodynamic diameters corresponding to the 50% percentile (Probit 5) to determine the MMAD, and the aerodynamic diameters corresponding the 15.87% percentile (Probit 4) and 84.13% percentile (Probit 6) to calculate the GSD. The results of this analysis are shown in Table 5-8 and FIG. 2, and are presented as mean±standard deviation (n=3)

TABLE 5
Aerosol performance of clofazimine-amikacin physical blends at 4 kPa pressure
drop through medium resistance RS01 device (amikacin component)

| | Jet milled | 1-0.5 CFZ-AMK | 1-2 CFZ-AMK | 1-4 CFZ-AMK | 1-8 CFZ-AMK |
|---|---|---|---|---|---|
| | AMK-S alone | physical blend | physical blend | physical blend | physical blend |
| EF (%) | 82.1 ± 1.2 | 80.0 ± 4.4 | 87.1 ± 2.0 | 88.4 ± 3.8 | 82.8 ± 0.5 |
| RF < 5 μm | 40.8 ± 7.2 | 47.2 ± 2.2 | 39.3 ± 6.2 | 44.7 ± 4.2 | 42.1 ± 1.1 |
| (%) | | | | | |
| RF < 3 μm | 28.2 ± 4.9 | 29.9 ± 4.5 | 22.9 ± 5.8 | 27.6 ± 3.3 | 27.2 ± 1.5 |
| (%) | | | | | |
| MMAD | 9.07 ± 1.76 | 7.65 ± 0.44 | 10.79 ± 3.45 | 8.45 ± 0.55 | 8.90 ± 0.28 |
| (μm) | | | | | |
| GSD (μm) | 1.40 ± 0.03 | 1.35 ± 0.18 | 1.39 ± 0.07 | 1.35 ± 0.02 | 1.38 ± 0.01 |

TABLE 6
Aerosol performance of clofazimine-amikacin physical blends at 4 kPa pressure
drop through medium resistance RS01 device (clofazimine component)

| | Jet milled CFZ | 1-0.5 CFZ-AMK | 1-2 CFZ-AMK | 1-4 CFZ-AMK | 1-8 CFZ-AMK |
|---|---|---|---|---|---|
| | alone | physical blend | physical blend | physical blend | physical blend |
| EF (%) | 76.2 ± 4.1 | 80.3 ± 1.6 | 85.9 ± 3.8 | 86.7 ± 5.2 | 82.6 ± 0.7 |
| RF < 5 μm | 58.8 ± 4.7 | 53.8 ± 3.2 | 53.5 ± 6.2 | 50.9 ± 3.8 | 54.5 ± 0l7 |
| (%) | | | | | |
| RF < 3 μm | 47.4 ± 3.7 | 34.4 ± 3.2 | 32.5 ± 5.1 | 31.5 ± 2.0 | 37.9 ± 1.1 |
| (%) | | | | | |
| MMAD | 5.36 ± 0.53 | 7.03 ± 0.51 | 7.50 ± 1.07 | 7.48 ± 0.74 | 6.52 ± 0.14 |
| (μm) | | | | | |
| GSD (μm) | 1.39 ± 0.03 | 1.35 ± 0.01 | 1.35 ± 0.04 | 1.34 ± 0.05 | 1.36 ± 0.01 |

TABLE 7
Aerosol performance of clofazimine-amikacin physical blends at 2 kPa pressure
drop through medium resistance RS01 device (amikacin component)

| | Jet milled | 1-0.5 CFZ-AMK | 1-2 CFZ-AMK | 1-4 CFZ-AMK | 1-8 CFZ-AMK |
|---|---|---|---|---|---|
| | AMK-S alone | physical blend | physical blend | physical blend | physical blend |
| EF (%) | 77.2 ± 11.2 | 78.2 ± 0.6 | 77.3 ± 5.4 | 79.3 ± 3.1 | 81.8 ± 1.6 |
| RF < 5 μm | 48.3 ± 3.8 | 40.5 ± 1.4 | 35.6 ± 3.1 | 39.7 ± 3.3 | 41.1 ± 3.1 |
| (%) | | | | | |
| RF < 3 μm | 30.4 ± 3.4 | 17.4 ± 2.1 | 16.2 ± 1.8 | 21.1 ± 3.3 | 23.6 ± 2.5 |
| (%) | | | | | |
| MMAD | 8.23 ± 0.57 | 9.31 ± 0.33 | 12.07 ± 2.17 | 10.55 ± 1.49 | 10.04 ± 0.94 |
| (μm) | | | | | |
| GSD (μm) | 1.37 ± 0.03 | 1.25 ± 0.00 | 1.36 ± 0.06 | 1.37 ± 0.04 | 1.40 ± 0.02 |

TABLE 8
Aerosol performance of clofazimine-amikacin physical blends at 2 kPa pressure
drop through medium resistance RS01 device (amikacin component)

| | Jet milled CFZ | 1-0.5 CFZ-AMK | 1-2 CFZ-AMK | 1-4 CFZ-AMK | 1-8 CFZ-AMK |
|---|---|---|---|---|---|
| | alone | physical blend | physical blend | physical blend | physical blend |
| EF (%) | 80.7 ± 9.7 | 73.8 ± 0.7 | 74.6 ± 7.5 | 77.0 ± 4.3 | 79.4 ± 6.9 |
| RF < 5 μm | 51.0 ± 19.0 | 46.9 ± 3.2 | 46.2 ± 0.7 | 49.3 ± 5.1 | 51.2 ± 14.1 |
| (%) | | | | | |
| RF < 3 μm | 37.5 ± 18.2 | 24.0 ± 3.2 | 23.5 ± 0.8 | 27.8 ± 6.0 | 31.7 ± 10.6 |
| (%) | | | | | |
| MMAD | 10.08 ± 7.79 | 8.91 ± 0.78 | 9.61 ± 0.62 | 8.17 ± 3.96 | 7.62 ± 1.82 |
| (μm) | | | | | |
| GSD (μm) | 1.46 ± 0.11 | 1.34 ± 0.01 | 1.37 ± 0.05 | 1.32 ± 0.01 | 1.36 ± 0.02 |

### C. Efficacy of Physically Blended CFZ and AMK in the Treatment of Intracellular Mab Infections

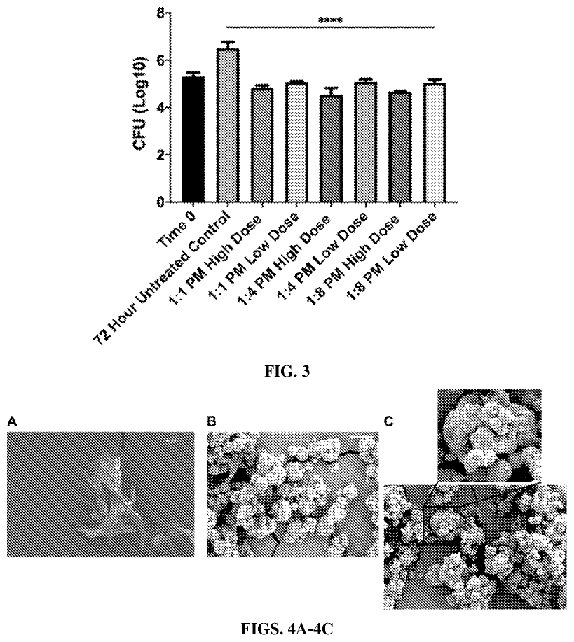

The efficacy of the physically blended CFZ-AMK formulations was assessed according to the steps below:
1. THP-1 monocytes were differentiated into macrophages using 15 ng/ml PMA for 72 hours.
2. Macrophages were infected with M. abscessus Rough morphotype (ATCC 19977) at a multiplicity of infection (MOI) of 10. The cells were incubated for 6 hours in antibiotic-free media, after which they were washed 3× with pre-warmed PBS to remove extracellular bacteria.
3. CFZ-AMK formulations were suspended in antibiotic-free media using a high shear rotar stator homogenizer in a sterile environment. The following formulations were assessed:
   a. 1:1 physical mixture of CFZ:AMK
   b. 1:4 physical mixture of CFZ:AMK
   c. 1:8 physical mixture of CFZ:AMK
4. The formulations were assessed at a “high dose”, e.g., 100 ug/mL powder added to the media and a “low dose”, e.g., 20 ug/mL powder added to the media.
5. The cell culture media was replaced every 24 hours with fresh CFZ-AMK containing media for a total of 72 hours.
6. After 72 hours, the media was aspirated, and cells were washed 2× with pre-warmed PBS to remove extracellular bacteria/particles. 1 mL of 0.1% Triton-X in PBS was added to lyse the cells
7. Varying dilutes of the cell lysates were plated on Middlebrook 7H10 agar plates supplemented with OADC and 0.4% activated charcoal and incubated for 3-7 days to enumerate colonies (FIG. 3).

An additional efficacy study was conducted following the steps below:
1. THP-1 monocytes seeded into 12 well plates at a density of 5×10⁵ cells/mL and were differentiated into macrophages using 15 ng/mL PMA for 72 hours
2. Macrophages were infected with M. abscessus Rough morphotype (ATCC 19977) at a multiplicity of infection (MOI) of 10. The cells were incubated for 3 hours in antibiotic-free media, after which they were washed 3× with pre-warmed PBS to remove extracellular bacteria.
3. CFZ-AMK formulations were suspended in antibiotic-free media using a high shear rotar stator homogenizer in a sterile environment, then diluted in culture media to achieve the desired concentrations. The following formulations were assessed:
   a. 1:0.5 physical mixture of CFZ:AMK, 250 ug/mL
   b. 1:2 physical mixture of CFZ:AMK, 250 ug/mL
   c. 1:4 physical mixture of CFZ:AMK, 250 ug/mL
   d. 1:8 physical mixture of CFZ:AMK, 250 ug/mL
   e. Air jet milled amikacin sulfate, 233 ug/mL (equivalent to highest concentration of amikacin free base tested in the blends)
   f. Air jet milled clofazimine, 145 ug/mL (equivalent to highest concentration of clofazimine tested in the blends)
4. An untreated time 0 control was collected to determine the infection baseline. Following that, timepoints were collected at 2 hours, 4 hours, 24 hours and 48 hours post treatment. An untreated 48 hour control was also collected. All treatment timepoints and untreated controls were comprised of n=3 wells.
5. At each timepoint, samples were collected as follows: the cell culture supernatant was removed and transferred to sterile microcentrifuge tube. The cells were washed 2× with pre-warmed PBS to remove extracellular bacteria/particles. 0.1 mL of 0.1% Triton-X in PBS was added to lyse the cells. The lysate was transferred to a sterile microcentrifuge
6. Serial dilutions of the cell lysates and supernatants were plated on Middlebrook 7H10 agar plates supplemented with OADC and 0.4% activated charcoal and incubated for 3-7 days to enumerate colonies See FIG. 4.

### Example 2: Generation of Spray Dried Composite Particles of Clofazimine and Amikacin

### Manufacturing of Powders:

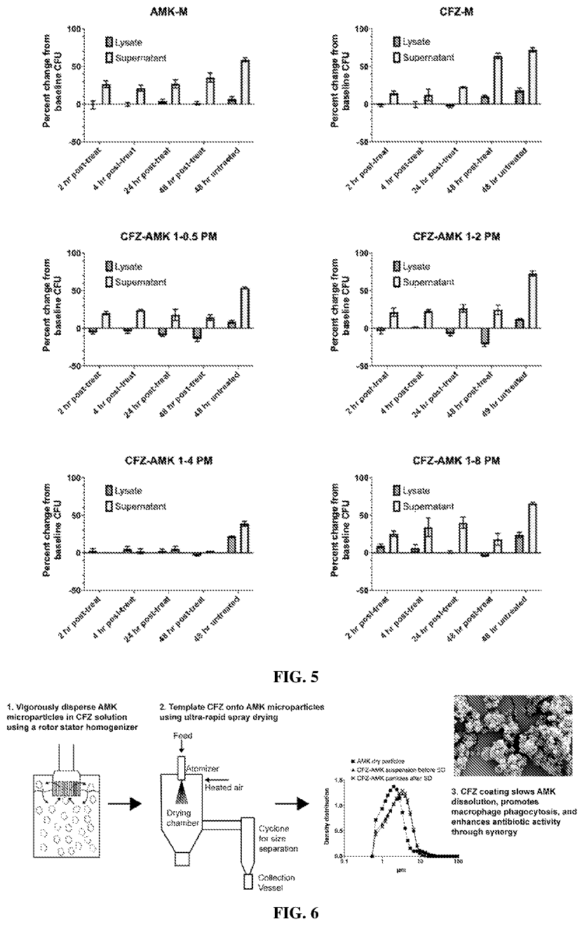

The opposing solubilities of CFZ (practically insoluble in water) and AMK sulfate (extremely water soluble, but insoluble in organic solvents) make the development of a co-formulated inhalation powder using particle engineering techniques such as spray drying extremely challenging. Furthermore, CFZ has the tendency to precipitate into needle-shaped crystals upon spray drying, which are unsuitable for macrophage uptake (Champion et al.) (FIG. 5). However, it was surprisingly observed that the utilization of an ultra-rapid feed flow rate (˜50 mL/min) of an feed stock consisting of micron-sized AMK particles suspended in an ethanol-based solution of CFZ results in the rapid precipitation of CFZ onto the surface of the AMK particles, without the formation of needle-crystals (FIG. 6). Historically, feed flow rates of 1-5 mL/min have been used for spray drying, as it is considered that this is necessary to achieve respirable particles.

This rapid precipitation onto the AMK particle surface is further enhanced by the low solubility of CFZ in ethanol and isopropanol (≤0.8 mg/mL). In essence, the AMK particles serve as template for the precipitation of CFZ, and virtually identical particle morphologies and size distributions have been observed at a variety of spray dryer atomization settings, which indicates the robustness of the process to manufacturing variability and amenability to scale-up operations. The incorporation of an extremely rapid feed flow rate into the process was further determined to reduce the need for stabilizing excipients in the feed stock, that would normally be necessary to ensure a uniform suspension of AMK particles during the spray drying process. Only a limited number of excipients are approved for the inhalation route, and the approval of new excipients requires extensive toxicity evaluation. Thus, the approach of developing a composite CFZ-AMK particles that are excipient-free or utilizing only excipients that are approved by the FDA for the oral inhalation route will significantly reduce development costs and de-risk regulatory approval. Further, the ratio of CFZ to AMK may be adjusted by increasing the concentration of AMK particles used in the processing method. A schematic of the processing method is shown in FIG. 6.

The generation of a two phase feed stock for spray drying can be achieved using anti-solvent precipitation or homogenization of previously prepared amikacin sulfate microparticles into a clofazimine suspension, followed by immediate spray drying.

A protocol for generating a two phase feed stock using antisolvent precipitation is described below. FIG. 7 details the setup of the antisolvent precipitation process
1. Add an ethanol solution containing 0.5 mg/mL clofazimine, 0.012 mg/mL polysorbate 80 and 0.5 mg/mL distearoylphosphatidylcholine (DSPC) to a side arm flask and completely dissolved to generate a clofazimine antisolvent solution. The side arm flask should be a volume that is 5× larger than the suspension volume
2. Attach a vacuum line to the side arm, add a stir bar to the solution and a place a rubber stopper with a hole in it to the top of the flask. An impinger (i.e., cut plastic pipette) should be passed through the stopper and the tip submerged in the antisolvent
3. The flask is placed on a stir-plate (e.g., Magnetic “6×6” stirrer, Cole-Palmer Instrument Company) and set to continuously stir at a moderate pace so that vortex is generated in the antisolvent solution
4. A nebuliser (e.g., EZ Breather Atomizer EZ-100, Nephron Pharmaceutical Corp.) containing 1.5 mg/mL amikacin sulfate solution in water at a volume that is 1/50 that of the anti-solvent solution volume (i.e., 4 mL amikacin sulfate solution to 200 mL of clofazimine anti-solvent solution) is placed at the entrance of the impinger
5. The vacuum should be turned on to draw nebulized amikacin sulfate droplets into the antisolvent solution below. The nebulizer is turned on. Hot air is applied to the length of the impinger using an air gun to prevent condensation of the nebulized droplets in the impinger line.
6. After the solution is nebulized, the generated suspension is sonicated for two minutes at 40 kHz in a sonicating bath (e.g., Branson 5800 Ultrasonic Cleaner).
7. If desired, additional particle size reduction can be obtained by running the suspension through a high-pressure homogenizer (e.g., Microfluidics M-110P) at 25000 PSI for 40 cycles.
8. The particle size distribution of the amikacin sulfate suspension phase is stable for up to 24 hours. Following generation of the suspension, the two-phase feed stock should be spray dried (e.g., using a BUCHI B-290 mini spray dryer with B-295 inert loop) at a feed rate of 50 mL/min, 22.9 L/min atomization air flow rate, inlet temperature of 140-150° C., and the maximum aspiration air flow available on the spray drier.

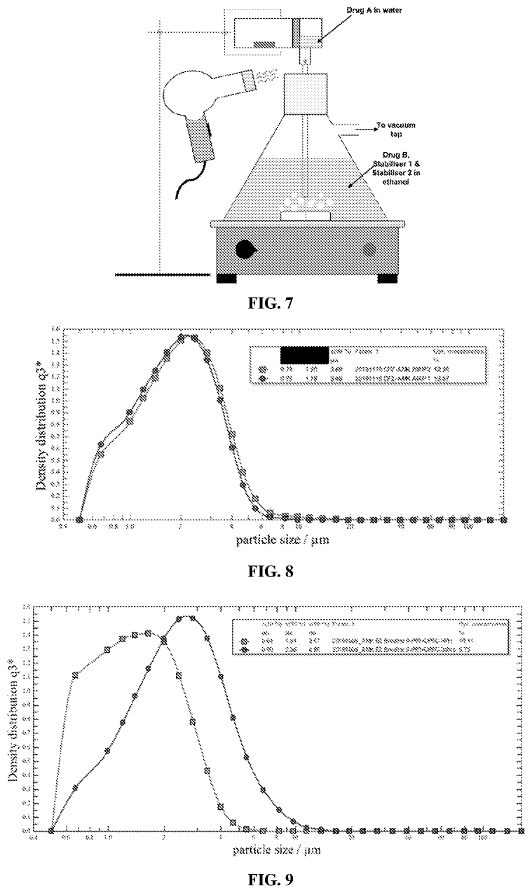

The particle size distribution of the amikacin sulfate component of the two-phase feed stock prepared using the anti-solvent precipitation is shown in FIG. 8. The suspension before and after high pressure homogenization is shown in FIG. 9.

A protocol for generating various ratios of composite CFZ-AMK spray dried particles using sequential spray drying is described below:
1. A 1% w/v aqueous solution of amikacin sulfate is spray dried at 130° C. inlet temperature, a 1 mL/min feed flow rate, and a 22.9 L/min atomization air flow rate. The collected powder is referred to as AMK-S-SD here on.
2. A 140 mL of 0.5 mg/mL CFZ in ethanol solution was prepared. The solution was sonicated and mixed for several minutes to ensure complete dissolution. It is very important the CFZ is completely dissolved.
3. Preparation of a 1:1 feed stock:
   a. First, 75 mg of AMK-S-SD (corresponds to 56 mg AMK free base) was suspended in 25 mL of 0.5 mg/mL CFZ solution. The suspension was homogenized using a rotor stator homogenizer to ensure complete dispersion of the AMK particles.
   b. Then, an additional 112 mL of CFZ 0.5 mg/mL solution was added
   c. Finally, an addition 87 mL of pure ethanol was used to wash the homogenizer blades and CFZ transfer container to ensure all drug was obtained.
4. Preparation of 1:4 feed stock:
   a. First, 75 mg of AMK-S-SD (corresponds to 56 mg AMK free base) was suspended in 25 mL of 0.5 mg/mL CFZ solution. The suspension was homogenized using a rotor stator homogenizer to ensure complete dispersion of the AMK particles.
   b. Then, an additional 3 mL of CFZ 0.5 mg/mL solution was added
   c. Finally, an additional 84 mL of pure ethanol was used to wash the homogenizer blades, CFZ transfer container, etc, to ensure all drug was obtained.
5. Preparation of 1:8 feed stock:
   a. First, 75 mg of AMK-S-SD (corresponds to 56 mg AMK free base) was suspended in 14 mL of 0.5 mg/mL CFZ solution+11 mL of pure ethanol. The suspension was homogenized using a rotor stator homogenizer to ensure complete dispersion of the AMK particles.
   b. Finally, an additional 87 mL of pure ethanol was used to wash the homogenizer blades, CFZ transfer container, etc, to ensure all drug was obtained.
6. Finally, the CFZ-AMK feed stocks were spray dried at 150° C. inlet temperature, with the atomization air rate set at 40 on the Buchi B290 rotometer. The peristaltic tubing from the atomizer was set directly in the flask containing the feed stock. The negative pressure generated from the atomization sucks up the feed stock at an approximately 50 mL/min feed rate.

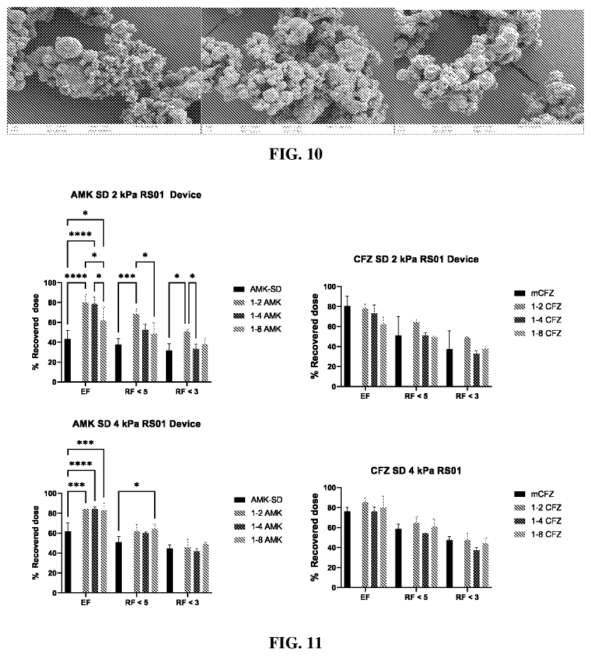

The resulting particle size distribution (PSD) of the spray dried powders are described in Table 9, while FIG. 10 shows the particle morphology of the formulations.

TABLE 9
Particle size distribution of CFZ-AMK spray dried particles

| Sample | X10 (μm) | X50 (μm) | X90 (μm) |
|---|---|---|---|
| 1:1 SD | 1.24 | 2.86 | 7.04 |
| 1:4 SD | 1.57 | 3.23 | 6.91 |
| 1:8 SD | 1.49 | 2.94 | 5.77 |

An additional protocol for generating various ratios of composite CFZ-AMK spray dried particles using sequential spray drying is described below:
7. A 1% w/v aqueous solution of amikacin sulfate is spray dried at 130° C. inlet temperature, a 1 mL/min feed flow rate, and a 22.9 L/min atomization air flow rate. The collected powder is referred to as AMK-S-SD here on.
8. Prepare a 0.75 mg/mL clofazimine solution in ethanol. The solution should be sonicated and mixed for several minutes to ensure complete dissolution. It is very important the clofazimine is completely dissolved.
9. Preparation of a 1:8 CFZ-AMK feed stock:
   a. Prepare an 8.3 mg/mL suspension of AMK-S-SD (equivalent to 6.0 mg/mL suspension of amikacin free base) in a volume of 0.75 mg/mL clofazimine solution that is equivalent to 12% of the desired final feed stock volume. The suspension should be homogenized using a rotor stator homogenizer to ensure complete dispersion of the AMK particles.
   b. Add an amount of pure isopropanol sufficient to bring the volume up to 22% of the desired final feed stock volume.
   c. Wash the blades of the homogenizer with pure isopropanol sufficient to bring the volume to the desired final feed stock volume
10. Preparation of a 1:4 CFZ-AMK feed stock
   a. Prepare a 4.5 mg/mL suspension of AMK-S-SD (equivalent to 3.2 mg/mL suspension of amikacin free base) in a volume of 0.75 mg/mL clofazimine solution that is equivalent to 22% of the desired final feed stock volume. The suspension should be homogenized using a rotor stator homogenizer to ensure complete dispersion of the AMK particles.
   b. Add an additional volume of clofazimine solution to bring the volume to 24% of the final volume
   c. Wash the homogenizer blades with pure isopropanol sufficient to bring the volume to the desired final feed stock volume.
11. Preparation of a 1:2 CFZ-AMK feed stock
   a. Prepare a 4.5 mg/mL suspension of AMK-S-SD (equivalent to 3.2 mg/mL suspension of amikacin free base) in a volume of 0.75 mg/mL clofazimine solution that is equivalent to 22% of the desired final feed stock volume. The suspension should be homogenized using a rotor stator homogenizer to ensure complete dispersion of the AMK particles.
   b. Add an additional volume of clofazimine solution to bring the volume to 47% of the desired final volume
   c. Wash the homogenizer blades with pure isopropanol sufficient to bring the volume to the desired final feed stock volume.
12. Preparation of a 1:0.5 CFZ-AMK feed stock
   a. In this case, the feed stock is prepared at a 1:0.25 CFZ-AMK ratio to account for loss of untemplated CFZ to the filter during the spray drying process
   b. Prepare a 1.1 mg/mL suspension of AMK-S-SD (equivalent to 0.79 mg/ml suspension of amikacin free base) in a volume of 0.75 mg/mL clofazimine solution that is equivalent to 22% of the desired final feed stock volume. The suspension should be homogenized using a rotor stator homogenizer to ensure complete dispersion of the AMK particles.
   c. Add an additional volume of clofazimine solution to bring the volume to 78% of the desired final volume
   d. Wash the homogenizer blades with pure isopropanol sufficient to bring the volume to the desired final feed stock volume.
13. Immediately after preparation of the two-phase feed stocks, the feed is spray dried at 150° C. inlet temperature, and 22.9 L/min atomization nitrogen flow rate using a two fluid nozzle with 0.7 mm nozzle diameter, and feed flow rate of 30 mL/min. This procedure may be performed using any standard spray dryer with an organic solvent trap (e.g., BUCHI B 290 mini spray dyer with B 295 inert loop; Pilotech YC 501).

The content uniformity of the clofazimine component of the powder was assessed by dissolving a known amount of the spray dried powder in a 75-25 mixture of acetonitrile and water containing 0.13 mg/mL polysorbate 80 and adding a volume of 5 M ammonium acetate equivalent to 16.6% of the original solution volume to induce phase separation. The upper organic phase was assayed for clofazimine and the lower aqueous phase assayed for amikacin using UV absorbance and concentration was quantified by using a standard curve of known spiked concentrations (which had undergone the same phase separation procedure). The results of the content uniformity assay are described in Table 10 below.

TABLE 10
Content Uniformity of CFZ-AMK composite spray dried particles

| | Average | Clofazimine | Average | Amikacin |
|---|---|---|---|---|
| | clofazimine | coefficient | amikacin | coefficient |
| Feed stock | content | of | content | of |
| Ratio | (%) | variation | (%) | variation |
| (CFZ:AMK) | (n = 5) | (%) | (n = 5) | (%) |
| 1:0.25 | 57.7 | 1.1 | 28.4 | 5.7 |
| 1:2 | 25.2 | 1.7 | 52.2 | 1.1 |
| 1:4 | 13.4 | 4.1 | 60.9 | 5.3 |
| 1:8 | 7.0 | 4.5 | 69.2 | 7.1 |

The aerosol performance of the composite spray dried particles was determined using cascade impaction, specifically as Next Generation Impactor, as prescribed by the United States Pharmacopeia chapter 601. For these studies, a medium resistance Model 7 RS01 dry powder inhaler (DPI) manufactured by Berry Global Osnago was utilized. 20 mg of the spray dried powders (1:2 CFZ-AMK, 1:4 CFZ-AMK, or 1:8 CFZ-AMK) or 10 mg of air jet milled clofazimine or spray dried amikacin sulfate alone was loaded into a size 3 hydroxypropyl methylcellulose (HPMC) capsule. For these studies, spray dried clofazimine was not tested due to the extremely low powder yield and the tendency of the particles to dry as large needle structures. The device was actuated at an airflow rate to induce a 4 kPa pressure drop (i.e., 80 L/min) or 2 kPa pressure drop (i.e., 56.6 L/min) through the device for period of time sufficient to draw 4 L of air through the apparatus. To reduce particle bounce and re-entrainment, the NGI plates were coated with 1% (v/v) glycerin in ethanol and allowed to dry prior to performing the experiment. After actuation, powder was collected from the capsule, device, induction port, and stages of the Next Generation Impactor by adding a volume of 75/25 acetonitrile/water containing 0.16 mg/mL polysorbate 80 that was sufficient to dissolve the deposited powder. Phase separation of the solution into an organic phase containing clofazimine and an aqueous phase containing amikacin was induced by adding a volume of 5 M ammonium acetate equivalent to 16.7% of the original solution volume. The organic and aqueous phases were analyzed using high pressure liquid chromatography and concentration of each drug was quantified using a previously prepared standard curve.

Stage 1-7 cut-off diameters were determined using equation 1 and MOC cut-off diameters were determined using equation 2.

$${D}_{50 , Q} = {{D}_{50 , Qn} ( \frac{{Q}_{n}}{Q} )}^{X} \qquad Eq ( 1 )$$

$${D}_{80 , Q} = 0.14 {( \frac{{Q}_{n}}{Q} )}^{1.36} \qquad Eq ( 2 )$$

where D_{50,Q} is the cutoff diameter at the flow rate, Q, and the subscript, n, refers to the archival reference value for Qₙ=60 L/min, and the values for the exponent, x, were determined by the archival NGI stage cut size-flow rate calculations, as determined by Marple et al.

The emitted fraction (EF) was calculated as the total drug emitted from the device as a percentage of the total mass of drug collected (i.e., recovered dose). The respirable fraction that was less than 5 μm aerodynamic diameter and less than 3 μm aerodynamic diameter corresponded to the percentage of the recovered dose predicted to have the aerodynamic diameter below 5 μm and 3 μm. The respirable fraction values were interpolated from a graph with the cumulative percentage of the emitted dose deposited downstream from an NGI stage as the ordinate and the particle cutoff size of that stage as the abscissa. For each sample, the mass median aerodynamic diameter (MMAD), which represents the mass-based median point of the aerodynamic particle size distribution (APSD), and geometric standard deviation (GSD), which represents the spread of the APSD, were determined by plotting the cumulative percentage of mass less than the stated aerodynamic size cut (expressed as Probits) against the aerodynamic diameter (log scale). Distributions were log normal. A linear regression was performed to determine the aerodynamic diameters corresponding to the 50% percentile (Probit 5) to determine the MMAD, and the aerodynamic diameters corresponding the 15.87% percentile (Probit 4) and 84.13% percentile (Probit 6) to calculate the GSD. The results of this analysis are shown in Tables 11-14 and FIG. 11, and are presented as mean±standard deviation (n=3)

TABLE 11
Aerosol performance of clofazimine-amikacin spray dried powder at 4 kPa pressure
drop through medium resistance RS01 device (amikacin component)

| | Spray dried | 1-2 CFZ-AMK | 1-4 CFZ-AMK | 1-8 CFZ-AMK |
|---|---|---|---|---|
| | AMK-S alone | physical blend | physical blend | physical blend |
| EF (%) | 61.9 ± 8.6 | 83.9 ± 0.3 | 84.5 ± 2.0 | 82.9 ± 7.0 |
| RF < 5 μm | 50.8 ± 5.8 | 61.9 ± 6.6 | 60.0 ± 1.2 | 64.5 ± 4.4 |
| (%) | | | | |
| RF < 3 μm | 44.5 ± 3.6 | 45.5 ± 8.5 | 41.9 ± 2.5 | 49.1 ± 2.0 |
| (%) | | | | |
| MMAD | 6.23 ± 0.97 | 5.57 ± 1.06 | 5.74 ± 0.22 | 5.13 ± 0.21 |
| (μm) | | | | |
| GSD (μm) | 1.58 ± 0.05 | 1.30 ± 0.04 | 1.25 ± 0.01 | 1.25 ± 0.01 |

TABLE 12
Aerosol performance of clofazimine-amikacin spray dried powder at 4 kPa pressure
drop through medium resistance RS01 device (clofazimine component)

| | Jet milled | 1-2 CFZ-AMK | 1-4 CFZ-AMK | 1-8 CFZ-AMK |
|---|---|---|---|---|
| | CFZ alone | physical blend | physical blend | physical blend |
| EF (%) | 76.2 ± 4.1 | 85.3 ± 4.0 | 76.2 ± 4.4 | 80.3 ± 11.0 |
| RF < 5 μm | 58.8 ± 4.7 | 64.6 ± 6.0 | 54.0 ± 0.3 | 60.7 ± 7.8 |
| (%) | | | | |
| RF < 3 μm | 47.4 ± 3.7 | 47.4 ± 7.0 | 37.4 ± 2.6 | 44.2 ± 5.0 |
| (%) | | | | |
| MMAD | 5.36 ± 0.53 | 5.38 ± 0.90 | 6.6 ± 0.4 | 5.50 ± 0.56 |
| (μm) | | | | |
| GSD (μm) | 1.39 ± 0.03 | 1.33 ± 0.05 | 1.33 ± 0.02 | 1.23 ± 0.05 |

TABLE 13
Aerosol performance of clofazimine-amikacin spray dried powder at 4 kPa pressure
drop through medium resistance RS01 device (clofazimine component)

| | Spray dried | 1-2 CFZ-AMK | 1-4 CFZ-AMK | 1-8 CFZ-AMK |
|---|---|---|---|---|
| | AMK-S alone | physical blend | physical blend | physical blend |
| EF (%) | 43.3 ± 8.7 | 80.2 ± 7.8 | 78.7 ± 6.6 | 61.7 ± 13.4 |
| RF < 5μm | 37.6 ± 6.2 | 68.5 ± 4.8 | 52.4 ± 6.0 | 48.6 ± 11.1 |
| (%) | | | | |
| RF < 3 μm | 31.8 ± 6.9 | 50.8 ± 2.1 | 33.6 ± 4.4 | 38.1 ± 5.7 |
| (%) | | | | |
| MMAD | 11.29 ± 2.93 | 5.81 ± 0.56 | 7.73 ± 0.63 | 8.02 ± 1.71 |
| (μm) | | | | |
| GSD (μm) | 1.62 ± 0.02 | 1.33 ± 0.08 | 1.29 ± 0.03 | 1.38 ± 0.09 |

TABLE 14
Aerosol performance of clofazimine-amikacin spray dried powder at 2 kPa pressure
drop through medium resistance RS01 device (clofazimine component)

| | Jet milled | 1-2 CFZ-AMK | 1-4 CFZ-AMK | 1-8 CFZ-AMK |
|---|---|---|---|---|
| | CFZ alone | physical blend | physical blend | physical blend |
| EF (%) | 80.7 ± 9.7 | 78.3 ± 4.2 | 73.3 ± 8.4 | 62.4 ± 7.2 |
| RF < 5 μm | 51.0 ± 19.0 | 64.6 ± 2.2 | 51.2 ± 2.8 | 49.2 ± 0.8 |
| (%) | | | | |
| RF < 3 μm | 37.5 ± 18.2 | 49.1 ± 0.8 | 32.9 ± 2.9 | 37.7 ± 1.8 |
| (%) | | | | |
| MMAD | 10.08 ± 7.79 | 5.58 ± 0.12 | 7.67 ± 0.39 | 7.45 ± 0.28 |
| (μm) | | | | |
| GSD (μm) | 1.46 ± 0.11 | 1.31 ± 0.02 | 1.32 ± 0.04 | 1.32 ± 0.04 |

### C. X-Ray Powder Diffraction Analysis of Spray Dried CFZ-AMK Particles

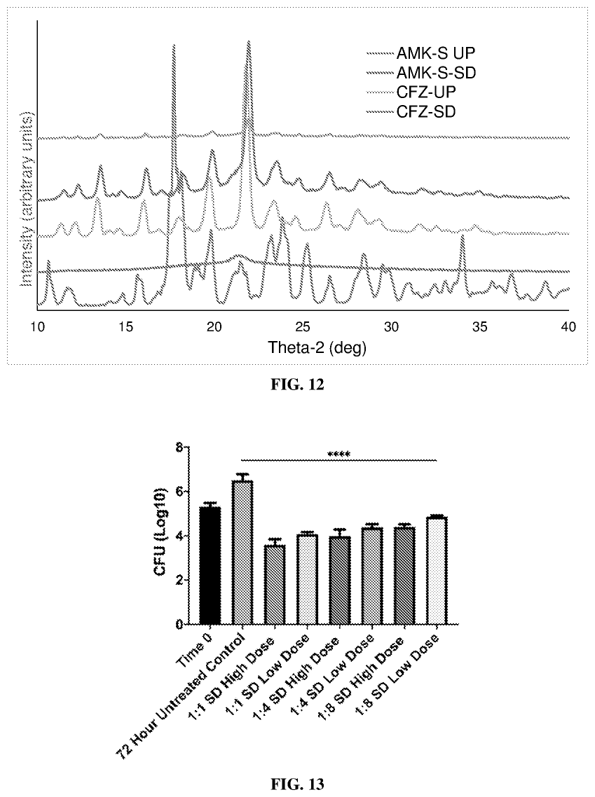

X-ray powder diffraction analysis of spray dried CFZ-AMK particles was conducted using a Rigaku Miniflex 600 Diffractometer II. The data was collected in continuous mode at a 2Θ range of 10-40°, with a 0.04° step size, speed of 1°/min with a target radiation of 40 kV and 15 mA. Diffraction patterns of unprocessed clofazimine, unprocessed amikacin sulfate, spray dried clofazimine, spray dried amikacin sulfate, and composite CFZ-AMK spray dried particles (1:2 ratio) was collected. The spectra indicates that the CFZ-AMK particles exhibit a partially crystalline structure with peaks corresponding to the unprocessed triclinic form of clofazimines (FIG. 12). No peaks corresponding to crystalline amikacin sulfate were observed. These data indicate that clofazimine is in a crystalline phase, while amikacin remains in an amorphous phase.

D. Efficacy of Spray Dried CFZ-AMK Particles in the Treatment of Intracellular Mab infections

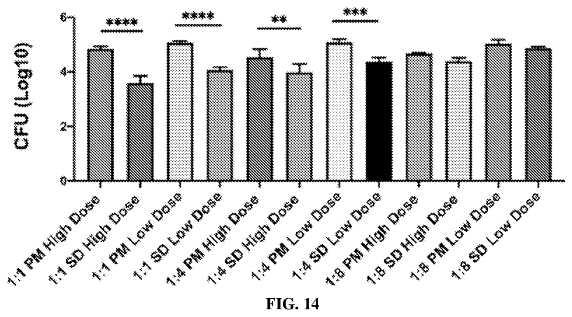

The efficacy of the spray dried CFZ-AMK formulations were assessed according to the steps below:
1. THP-1 monocytes were differentiated into macrophages using 15 ng/mL PMA for 72 hours.
2. Macrophages were infected with M. abscessus Rough morphotype at a multiplicity of infection (MOI) of 10. The cells were incubated for 6 hours in antibiotic-free media, after which they were washed 3× with pre-warmed PBS to remove extracellular bacteria.
3. CFZ-AMK formulations were suspended in antibiotic-free media using a high shear rotar stator homogenizer in a sterile environment. The following formulations were assessed:
4. Composite spray dried particles prepared at a ratio of 1:1 CFZ:AMK
5. Composite spray dried particles prepared at a ratio of 1:4 CFZ:AMK
6. Composite spray dried particles prepared at a ratio of 1:8 CFZ:AMK
7. The formulations were assessed at a “high dose”, i.e., 100 ug/mL powder added to the media and a “low dose”, i.e., 20 ug/mL powder added to the media.
8. The cell culture media was replaced every 24 hours with fresh CFZ-AMK containing media for a total of 72 hours.
9. After 72 hours, the media was aspirated, and cells were washed 2× with pre-warmed PBS to remove extracellular bacteria/particles. 1 mL of 0.1% Triton-X in PBS was added to lyse the cells
10. Varying dilutes of the cell lysates were plated on Middlebrook 7H10 agar plates supplemented with OADC and 0.4% activated charcoal and incubated for 3-7 days to enumerate colonies (FIGS. 13 & 14).

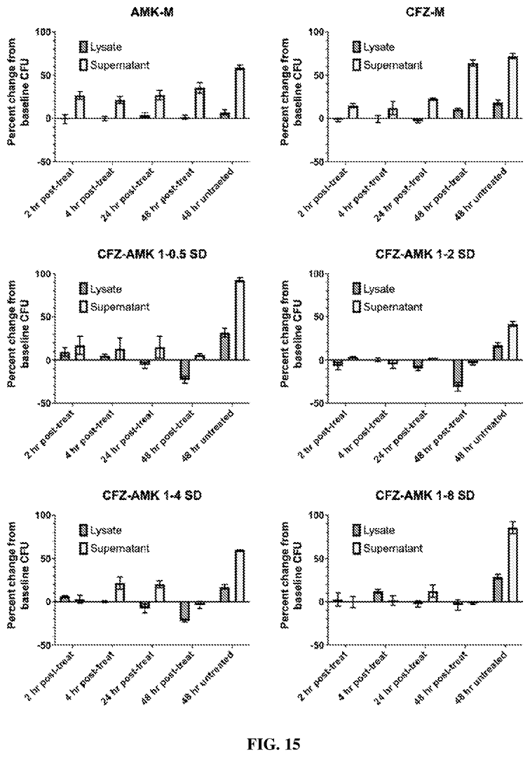

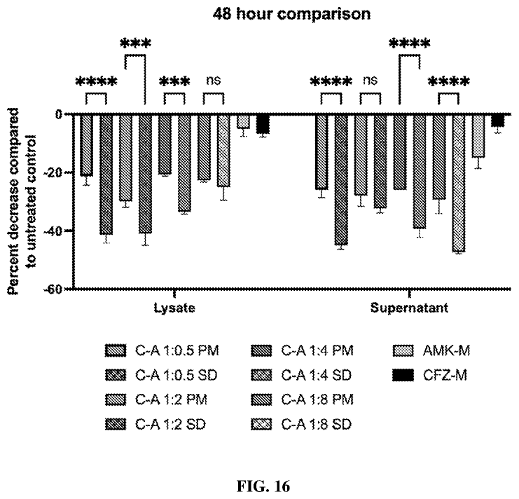

An additional efficacy study was conducted following the steps below:
1. THP-1 monocytes seeded into 12 well plates at a density of 5×10⁵ cells/mL and were differentiated into macrophages using 15 ng/mL PMA for 72 hours
2. Macrophages were infected with M. abscessus Rough morphotype (ATCC 19977) at a multiplicity of infection (MOI) of 10. The cells were incubated for 3 hours in antibiotic-free media, after which they were washed 3× with pre-warmed PBS to remove extracellular bacteria.
3. CFZ-AMK formulations were suspended in antibiotic-free media using a high shear rotar stator homogenizer in a sterile environment, then diluted in culture media to achieve the desired concentrations. The following formulations were assessed:
   a. 1:0.5 CFZ:AMK-SD, 250 ug/mL
   b. 1:2 CFZ:AMK-SD, 250 ug/mL
   c. 1:4 CFZ:AMK-SD, 250 ug/mL
   d. 1:8 CFZ:AMK-SD, 250 ug/mL
   e. Air jet milled amikacin sulfate, 233 ug/mL (equivalent to highest concentration of amikacin free base tested in the blends)
   f. Air jet milled clofazimine, 145 ug/mL (equivalent to highest concentration of clofazimine tested in the blends)
4. An untreated time 0 control was collected to determine the infection baseline. Following that, timepoints were collected at 2 hours, 4 hours, 24 hours and 48 hours post treatment. An untreated 48 hour control was also collected. All treatment timepoints and untreated controls were comprised of n=3 wells.
5. At each timepoint, samples were collected as follows: the cell culture supernatant was removed and transferred to sterile microcentrifuge tube. The cells were washed 2× with pre-warmed PBS to remove extracellular bacteria/particles. 0.1 mL of 0.1% Triton-X in PBS was added to lyse the cells. The lysate was transferred to a sterile microcentrifuge
6. Serial dilutions of the cell lysates and supernatants were plated on Middlebrook 7H10 agar plates supplemented with OADC and 0.4% activated charcoal and incubated for 3-7 days to enumerate colonies (FIGS. 15 & 16)

Surprisingly, the CFZ-AMK-SD particles resulted in statistically significant improvements in intracellular antibacterial efficacy compared to equivalent doses of physically blended formulations. These results indicate that the synergetic activity has been directed to the intracellular environment. Extracellular efficacy is also noted for several of the formulations.

### B. Aerosol Performance of Clofazimine and Amikacin Co-Administered Using Dual Chamber Inhaler Device

The aerosol performance of the air jet milled clofazimine and amikacin particles administered simultaneously from two different dosing chambers of a dual chamber device was determined using cascade impaction, specifically as Next Generation Impactor, as prescribed by the United States Pharmacopeia chapter 601. For these studies, a Dualhaler dry powder inhaler (DPI) manufactured by Emphasys was utilized. Varying weights of air jet milled clofazimine or air jet milled amikacin sulfate alone were individually loaded into a size 3 hydroxypropyl methylcellulose (HPMC) capsule and added to each side of the device to achieve the desired administration ratios (1:0.5 CFZ-AMK, 1:2 CFZ-AMK, 1:4 CFZ-AMK, 1:8 CFZ-AMK). The device was actuated at an airflow rate to induce a 4 kPa pressure drop (i.e., 80 L/min) or 2 kPa pressure drop (i.e., 56.6 L/min) through the device for period of time sufficient to draw 4 L of air through the apparatus. To reduce particle bounce and re-entrainment, the NGI plates were coated with 1% (v/v) glycerin in ethanol and allowed to dry prior to performing the experiment. After actuation, powder was collected from the capsule, device, induction port, and stages of the Next Generation Impactor by adding a volume of 75/25 acetonitrile/water containing 0.16 mg/mL polysorbate 80 that was sufficient to dissolve the deposited powder. Phase separation of the solution into an organic phase containing clofazimine and an aqueous phase containing amikacin was induced by adding a volume of 5 M ammonium acetate equivalent to 16.7% of the original solution volume. The organic and aqueous phases were analyzed using high pressure liquid chromatography and concentration of each drug was quantified using a previously prepared standard curve.

Stage 1-7 cut-off diameters were determined using equation 1 and MOC cut-off diameters were determined using equation 2.

$${D}_{50 , Q} = {{D}_{50 , Qn} ( \frac{{Q}_{n}}{Q} )}^{X} \qquad Eq ( 1 )$$

$${D}_{80 , Q} = 0.14 {( \frac{{Q}_{n}}{Q} )}^{1.36} \qquad Eq ( 2 )$$

where D_{50,Q} s the cutoff diameter at the flow rate, Q, and the subscript, n, refers to the archival reference value for Qₙ=60 L/min, and the values for the exponent, x, were determined by the archival NGI stage cut size-flow rate calculations, as determined by Marple et al.

The emitted fraction (EF) was calculated as the total drug emitted from the device as a percentage of the total mass of drug collected (i.e., recovered dose). The respirable fraction that was less than 5 μm aerodynamic diameter and less than 3 μm aerodynamic diameter corresponded to the percentage of the recovered dose predicted to have the aerodynamic diameter below 5 μm and 3 μm. The respirable fraction values were interpolated from a graph with the cumulative percentage of the emitted dose deposited downstream from an NGI stage as the ordinate and the particle cutoff size of that stage as the abscissa. The results of this analysis are shown in Tables 15-18 and are presented as mean±standard deviation (n=3), unless otherwise specified. For most of these experiments, the MMAD and GSD could not be calculated and are therefore not shown in the tables.

TABLE 15
Aerosol performance of clofazimine-amikacin physical blends at 4 kPa pressure
drop through medium resistance Dualhaler device (amikacin component)

| | Jet milled | | | | |
|---|---|---|---|---|---|
| | AMK-S alone | 1-0.5 | 1-2 | 1-4 | 1-8 |
| | (n = 2) | CFZ-AMK | CFZ-AMK | CFZ-AMK | CFZ-AMK |
| EF (%) | 47.8 ± 13.8 | 40.1 ± 18.3 | 50.5 ± 10.7 | 42.6 ± 17.0 | 48.3 ± 23.1 |
| RF < 5 μm | 28.6 ± 1.5 | 23.0 ± 19.6 | 22.0 ± 4.3 | 19.8 ± 7.2 | 24.8 ± 11.5 |
| (%) | | | | | |
| RF < 3 μm | 22.6 ± 1.4 | 15.8 ± 14.7 | 13.0 ± 4.2 | 13.0 ± 4.9 | 15.7 ± 6.7 |
| (%) | | | | | |

TABLE 16
Aerosol performance of clofazimine-amikacin physical blends at 4 kPa pressure
drop through medium resistance Dualhaler device (clofazimine component)

| | Jet milled CFZ | 1-0.5 CFZ-AMK | 1-2 CFZ-AMK | 1-4 CFZ-AMK | 1-8 CFZ-AMK |
|---|---|---|---|---|---|
| | alone | physical blend | physical blend | physical blend | physical blend |
| EF (%) | 44.9 ± 16.1 | 54.4 ± 5.2 | 45.6 ± 30.5 | 46.3 ± 9.8 | 44.7 ± 21.8 |
| RF < 5 μm | 30.8 ± 12.6 | 36.9 ± 2.6 | 31.9 ± 22.2 | 31.6 ± 8.2 | 30.4 ± 12.6 |
| (%) | | | | | |
| RF < 3 μm | 25.1 ± 10.9 | 28.8 ± 1.4 | 25.6 ± 18.6 | 24.5 ± 6.9 | 24.7 ± 10.3 |
| (%) | | | | | |

TABLE 17
Aerosol performance of clofazimine-amikacin physical blends at 2 kPa pressure
drop through medium resistance Dualhaler device (amikacin component)

| | Jet milled | 1-0.5 CFZ-AMK | 1-2 CFZ-AMK | 1-4 CFZ-AMK | 1-8 CFZ-AMK |
|---|---|---|---|---|---|
| | AMK-S alone | physical blend | physical blend | physical blend | physical blend |
| EF (%) | 14.1 ± 23.7 | 48.3 ± 19.8 | 22.9 ± 13.4 | 33.3 | 42.6 ± 16.0 |
| RF < 5 μm | 23.4 ± 4.6 | 29.4 ± 10.4 | 13.2 ± 5.7 | 24.9 | 20.3 ± 11.4 |
| (%) | | | | | |
| RF < 3 μm | 15.5 ± 2.4 | 19.5 ± 5.2 | 8.3 ± 3.2 | 16.4 | 12.3 ± 6.0 |
| (%) | | | | | |

TABLE 18
Aerosol performance of clofazimine-amikacin physical blends at 2 kPa pressure
drop through medium resistance Dualhaler device (amikacin component)

| | Jet milled | 1-0.5 CFZ-AMK | 1-2 CFZ-AMK | 1-4 CFZ-AMK | 1-8 CFZ-AMK |
|---|---|---|---|---|---|
| | CFZ alone | physical blend | physical blend | physical blend | physical blend |
| EF (%) | 41.2 ± 4.1 | 34.3 ± 16.3 | 40.7 ± 5.7 | 50.5 | 58.2 ± 16.1 |
| RF < 5 μm | 26.1 ± 3.1 | 22.7 ± 10.4 | 28.1 ± 4.8 | 37.4 | 30.1 ± 6.1 |
| (%) | | | | | |
| RF < 3 μm | 19.8 ± 2.2 | 16.0 ± 6.8 | 21.5 ± 4.3 | 30.0 | 22.5 ± 3.8 |
| (%) | | | | | |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Aldridge et al., Science, 335(6064): 100, 2012.
Arbiser et al., Journal of the American Academy of Dermatology, 32(2, Part 1):241-7, 1995.
Banaschewski et al., J Cyst Fibros., 18(5):714-20, 2019.
Bernut, et al., Proc Natl Acad Sci USA., 111(10):E943-52, 2014.
Bryant et al., Science, 354(6313):751-7, 2016.
Champion et al., Pharm Res., 26(1):244-9, 2009.
Connolly et al., PLOS Med., 4(3):e120, 2007.
Crowle et al., Infection and Immunity, 59(5): 1823-31, 1991.
Daley et al., Clin Infect Dis., 2020.
Dartois, Nat Rev Microbiol., 12(3): 159-67, 2014.
Esther et al., J Cyst Fibros. 9(2):117-23, 2010.
Flores et al., Microbiology, 151(2):521-32, 2005.
Frehel et al., Infection and Immunity, 52(1):252-62, 1986.
Hmama et al., Immunological Reviews, 264(1):220-32, 2015.
Holdiness Clinical Pharmacokinetics, 16(2):74-85, 1989.
Luthra et al., Front Microbiol., 9:2179, 2018.
Marple et al., J. Aerosol Medicine, 16:301-324, 2003.
Olivier et al., Am J Respir Crit Care Med., 195(6):814-23, 2017.
Pieters, Cell Host Microbe, 3(6): 399-407, 2008.
Roux et al., Open Biol., 6(11), 2016.
Russell et al., Nat Immunol., 10(9):943-8, 2009.
Sabnis, Inhaled Clofazimine Delivery for the Treatment of Pulmonary Tuberculosis, Master's Thesis, Creighton University, 2015.
Shen et al., Int J Antimicrob Agents, 35(4):400-4, 2010.
Sturgill-Koszycki et al., Science, 263(5147):678-81, 1994.
Swanson et al., Antimicrob Agents Chemother., 59(6):3042-51, 2015.
Szeto et al., Open Forum Infect Dis., 3(3):ofw004, 2016.
Tomashefski et al., American Journal of Respiratory and Critical Care Medicine, 154(2):523-8, 1996.
Zhang et al., Front Microbiol., 9:915, 2018.
Van Deun et al., Am J Respir Crit Care Med., 182(5):684-92, 2010.
van Dorn, The Lancet Respiratory Medicine, 5(1):15, 2017.
van Ingen et al., Antimicrob Agents Chemother., 56(12):6324-7, 2012.
Verma et al., Antimicrob. Agents Chemother., 57:1050-52, 2013.

1. A pharmaceutical composition comprising a combined dose of:
(A) clofazimine or a pharmaceutically acceptable salt; and
(B) amikacin or a pharmaceutically acceptable salt thereof, wherein the pharmaceutical composition is formulated for administration via inhalation.

2-3. (canceled)

4. The pharmaceutical composition of claim 1, wherein the clofazimine has been processed before mixing with amikacin by milling.

5. The pharmaceutical composition of claim 1, wherein the amikacin has been processed before mixing with clofazimine by milling.

6. (canceled)

7. The pharmaceutical composition of claim 1, wherein the amikacin and the clofazimine have been processed in a single mixture by spray drying.

8. (canceled)

9. The pharmaceutical composition according to claim 1, wherein the clofazimine is deposited on the surface of the amikacin.

10. The pharmaceutical composition according to claim 1, wherein less than 25% of the clofazimine is present as a crystal with an aspect ratio of not less than 0.75.

11-14. (canceled)

15. The pharmaceutical composition according to claim 1, wherein the amikacin is in the amorphous form.

16. The pharmaceutical composition of claim 15, wherein the amikacin is in the amorphous form and the clofazimine is in the crystalline form.

17. The pharmaceutical composition according to claim 1, wherein the pharmaceutical composition is formulated without any excipients.

18. The pharmaceutical composition according to claim 1, wherein the pharmaceutical composition is formulated with one or more excipients.

19. (canceled)

20. The pharmaceutical composition of claim 18, comprising a surfactant.

21-29. (canceled)

30. The pharmaceutical composition of claim 20, further comprising a lipid.

31-43. (canceled)

44. The pharmaceutical composition of claim 1, further comprising an ammonium salt.

45-53. (canceled)

54. The pharmaceutical composition according to claim 1, wherein the pharmaceutical composition has an X10 particle size distribution from about 0.25 μm to about 2.5 μm.

55-72. (canceled)

73. The pharmaceutical composition according to claim 1, wherein less than 50% of the pharmaceutical composition is dissolved at pH 7 within 6 hours.

74-75. (canceled)

76. The pharmaceutical composition according to claim 1, wherein more than 25% of the pharmaceutical composition is dissolved at pH 4.5 within 24 hours.

77-78. (canceled)

79. A method of preparing a pharmaceutical composition comprising:
(A) obtaining a clofazimine solution containing clofazimine in a solvent;
(B) admix amikacin particles to the clofazimine solution to obtain a two-phase mixture;
(C) spray drying the two-phase mixture to obtain a pharmaceutical composition;
wherein the spray drying has a feed rate of greater than 10 mL/min.

80-135. (canceled)

136. A pharmaceutical composition prepared according to claim 79.

137. A method of treating or preventing a lung disease comprising administering to a patient in need thereof a therapeutically effective amount of a pharmaceutical composition according to claim 1 anyone of claim 1.

138-152. (canceled)

153. An inhaler comprising:
(A) a device capable of delivering a dose of one or more therapeutic agents;
(B) clofazimine; and
(C) amikacin
wherein the clofazimine and the amikacin are formulated to be delivered in the inhaler.

154-159. (canceled)
